(19) United States
(12) Patent Application Publication
(10) Pub. No.: US 2014/0213575 A1
(43) Pub. Date: Jul. 31, 2014

(54) 6H-Thieno[3,2-f][1,2,4]triazolo[4,3-a][1,4]diazepines

(71) Applicants: Norbert Schmees, Berlin (DE); Joachim Kuhnke, Potsdam (DE); Bernard Haendler, Berlin (DE); Philip Lienau, Berlin (DE); Amaury Ernesto Fernandez-Montalvan, Berlin (DE); Pascale Lejeune, Berlin (DE); Stephan Siegel, Berlin (DE); William Scott, Guilford, CT (US)

(72) Inventors: Norbert Schmees, Berlin (DE); Joachim Kuhnke, Potsdam (DE); Bernard Haendler, Berlin (DE); Philip Lienau, Berlin (DE); Amaury Ernesto Fernandez-Montalvan, Berlin (DE); Pascale Lejeune, Berlin (DE); Stephan Siegel, Berlin (DE); William Scott, Guilford, CT (US)

(73) Assignee: BAYER INTELLECTUAL PROPERTY GMBH, Monheim (DE)

(21) Appl. No.: 14/342,519

(22) PCT Filed: Aug. 27, 2012
(86) PCT No.: PCT/EP2012/066600

§ 371 Date: Mar. 3, 2014

(30) Foreign Application Priority Data
Sep. 1, 2011 (DE) 102011082013.2

(51) Int. Cl. C07D 519/00

(52) U.S. Cl. CPC C07D 519/00
USPC 514/210.18; 540/560; 514/220; 540/543

(57) ABSTRACT

The invention relates to 6H-thieno[3,2-f][1,2,4]triazolo[4,3-a][1,4]diazepines, in particular for therapeutic purposes, pharmaceutical agents and use thereof in therapy, in particular for the prevention and treatment of tumour diseases.

The present invention relates to novel 6H-thieno[3,2-f][1,2,4]triazolo[4,3-a][1,4]diazepines, in particular for therapeutic purposes, pharmaceutical agents containing the compounds according to the invention and use thereof in therapy, in particular for the prevention and/or treatment of tumour diseases.

### BIOLOGICAL BACKGROUND

The human BET family (bromodomain and extra C-terminal domain family) has four members (BRD2, BRD3, BRD4 and BRDT), which contain two related bromodomains and one extraterminal domain (Wu and Chiang, J. Biol. Chem., 2007, 282:13141-13145). The bromodomains are protein regions that recognize acetylated lysine residues. These acetylated lysines are often found at the N-terminal end of histones (e.g. histone 3 or histone 4) and are characteristic features of an open chromatin structure and active gene transcription (Kuo and Allis, Bioessays, 1998, 20:615-626). In addition, bromodomains can recognize other acetylated proteins. For example, BRD4 binds to RelA, which leads to stimulation of NF-κB and transcriptional activity of inflammatory genes (Huang et al., Mol. Cell. Biol., 2009, 29:1375-1387). The extraterminal domain of BRD2, BRD3 and BRD4 interacts with several proteins having a role in chromatin modulation and regulation of gene expression (Rahman et al., Mol. Cell. Biol., 2011, 31:2641-2652).

Mechanistically BET proteins play an important role in cell growth and in the cell cycle. They are associated with mitotic chromosomes, suggesting a role in epigenetic memory (Dey et al., Mol. Biol. Cell, 2009, 20:4899-4909; Yang et al., Mol. Cell. Biol., 2008, 28:967-976). BRD4 is essential for transcription elongation and recruits the elongation complex P-TEFb, which consists of CDK9 and cyclin T1, which leads to activation of RNA polymerase II (Yang et al., Mol. Cell, 2005, 19:535-545). Consequently there is stimulation of the expression of genes that are involved in cell proliferation, such as c-Myc and Aurora B for example (You et al., Mol. Cell. Biol., 2009, 29:5094-5103; Zuber et al., Nature, 2011, doi: 10.1038). BRD2 and BRD3 bind to transcribed genes in hyperacetylated chromatin regions and promote transcription by RNA polymerase II (LeRoy et al., Mol. Cell, 2008, 30:51-60).

The knock-down of BRD4 in various cell lines leads to G1 arrest (Mochizuki et al., J. Biol. Chem., 2008, 283:9040-9048). It has also been shown that BRD4 binds to promoter regions of several genes that are activated in the G1 phase, for example cyclin D1 and D2 (Mochizuki et al., J. Biol. Chem., 2008, 283:9040-9048).

BRD2 and BRD4 knockout mice die early during embryogenesis (Gyuris et al., Biochim. Biophys. Acta, 2009, 1789:413-421; Houzelstein et al., Mol. Cell. Biol., 2002, 22:3794-3802). Heterozygous BRD4 mice have various growth defects, which can be attributed to reduced cellular proliferation (Houzelstein et al., Mol. Cell. Biol., 2002, 22:3794-3802).

BET proteins play an important role in various types of tumours. Fusion between the BET proteins BRD3 or BRD4 and NUT, a protein that normally is only expressed in the testis, leads to an aggressive form of squamous cell carcinoma, called NUT midline carcinoma (French, Cancer Genet. Cytogenet., 2010, 203:16-20). The fusion protein prevents cellular differentiation and promotes proliferation (Yan et al., J. Biol. Chem., 2011, 286:27663-27675). The growth of in vivo models derived therefrom is inhibited by a BRD4-inhibitor (Filippakopoulos et al., Nature, 2010, 468:1067-1073). Screening for therapeutic targets in an acute myeloid leukaemia cell line (AML) showed that BRD4 plays an important role in this tumour (Zuber et al., Nature, 2011, doi:10.1038). The reduction of BRD4 expression leads to selective arrest of the cell cycle and to apoptosis. Treatment with a BRD4-inhibitor prevents the proliferation of an AML xenograft in vivo. Amplification of the DNA region that contains the BRD4 gene was detected in primary breast tumours (Kadota et al., Cancer Res, 2009, 69:7357-7365). There are also data for BRD2 regarding a role in tumours. A transgenic mouse that overexpresses BRD2 selectively in B cells develops B cell lymphomas and leukaemias (Greenwall et al., Blood, 2005, 103:1475-1484). BET proteins are also involved in viral infections. BRD4 binds to the E2 protein of various papillomaviruses and is important for the survival of the viruses in latently infected cells (Wu et al., Genes Dev., 2006, 20:2383-2396). The herpesvirus that is responsible for Kaposi's sarcoma also interacts with various BET proteins, which is important for disease resistance (Viejo-Borbolla et al., J. Virol., 2005, 79:13618-13629; You et al., J. Virol., 2006, 80:8909-8919). By binding to P-TEFb, BRD4 also plays an important role in HIV replication (Bisgrove et al., Proc. Natl. Acad. Sci. USA, 2007, 104:13690-13695).

BET proteins are in addition involved in inflammatory processes. BRD2-hypomorphic mice display reduced inflammation in fat tissue (Wang et al., Biochem. J., 2009, 425:71-83). The infiltration of macrophages in white fat tissue is also reduced in BRD2-deficient mice (Wang et al., Biochem. J., 2009, 425:71-83). It has also been shown that BRD4 regulates a number of genes that are involved in inflammation. In LPS-stimulated macrophages, a BRD4-inhibitor prevents the expression of inflammatory genes, for example IL-1 or IL-6 (Nicodeme et al., Nature, 2010, 468:1119-1123).

These data provide evidence that the BET proteins play an essential role in various pathologies. It is therefore important to find potent and selective inhibitors that prevent interaction between the BET proteins and acetylated proteins. These new inhibitors should also have suitable pharmacokinetic properties that make it possible to inhibit these interactions in patients.

Examination of the structural state of the art is based on the following numbering for the ring system:

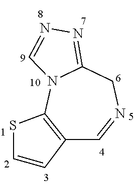 [SMILES: C1=NCc2nncn2-c2sccc21]

EP0638560 discloses, among other things, 6H-thieno[3,2-f][1,2,4]triazolo[4,3-a][1,4]diazepines for the treatment of osteoporosis. Substituted esters and amides are also provided in position 6 of the ring system, and no bridged elements or spiro elements are disclosed as substituents.

U.S. Pat. No. 5,712,274 discloses 6H-thieno[3,2-f][1,2,4]triazolo[4,3-a][1,4]diazepines for the treatment of inflammations. Amides substituted with heterocycles are also provided in position 6 of the ring system, or ring closures via the amide nitrogen. Example 50 discloses, for example, ring closure via the amide nitrogen to morpholine. Bridged elements or spiro elements are not included or disclosed. Inhibitory effects on proteins of the BRD family or possible use in cancers are not disclosed for the structures of U.S. Pat. No. 5,712,274.

EP0934940 discloses 6H-thieno[3,2-f][1,2,4]triazolo[4,3-a][1,4]diazepines for the treatment of inflammations. Substituted esters and amides are also provided in position 6 of the ring system, and no bridged elements or spiro elements are disclosed as substituents.

EP0989131 claims 6H-thieno[3,2-f][1,2,4]triazolo[4,3-a][1,4]diazepines bearing a carboxyalkyl side chain with amide function in position 6 of the ring system, in which the nitrogen atom bears a hydrogen atom and the residue R³. R³ can also represent an aromatic or heteroaromatic residue. Heterocycles via the amide nitrogen, bridged elements or spiro elements are not envisaged as meanings for R³. The compounds are disclosed for use in inflammatory and in allergic diseases, in which cell adhesion plays a role.

EP1887008 discloses 6H-thieno[3,2-f][1,2,4]triazolo[4,3-a][1,4]diazepines with substituted C₁-C₆ alkyl esters in position 6 of the ring system, wherein the alkyl ester is bound via an alkylene group to the ring system. Heterocycles such as morpholine are also envisaged as substituents of the alkyl ester. Amides in position 6, ring closures via the amide nitrogen, bridged elements or spiro elements are not included or disclosed. The application of the compounds described is in the area of inflammatory diseases.

EP2239264 discloses 6H-thieno[3,2-f][1,2,4]triazolo[4,3-a][1,4]diazepines for the treatment of cancers. The mechanism of action is stated to be inhibition of the BRD protein family. Primary amides are envisaged exclusively in position 6 of the ring system (R⁴), i.e. the amide nitrogen bears a hydrogen atom. R⁹ is a possible substituent of the amide nitrogen.

However, R⁹ does not comprise the meaning of bridged elements, spiro elements or ring closures via the amide nitrogen.

In Nature 2010, Vol. 468, p 1067ff (P. Filippakopoulos et al.), JQ-1 is described, which acts as strong binder to the BET protein family and has anti-proliferative properties, mediated thereby. JQ1 is comparative example V1 in the present application. The applicant regards JQ1 as the nearest prior art, as JQ1 relates to the same target and the same indications as the substances according to the invention.

WO2011/054553, WO2011/054843, WO2011/054844 and WO2011/054845 disclose 4H-[1,2,4]triazolo[4,3-a][1,4]benzodiazepines as bromodomain inhibitors.

The disclosure of WO2011/054553 relates to an individual benzodiazepine and use thereof in a wide variety of diseases.

Thieno[3,2-f][1,2,4]triazolo[4,3-a][1,4]diazepines are not disclosed.

The disclosure of WO2011/054843 relates to various individual substances, also including a benzodiazepine, and use thereof in inflammatory diseases or autoimmune diseases. Thieno[3,2-f][1,2,4]triazolo[4,3-a][1,4]diazepines are not disclosed.

The compounds according to the invention differ from the compounds of WO2011/054844 in that the obligate primary amide residue in position 6 of the ring system is bound directly to the diazepine ring and not via a methylene group. Bridged elements or spiro elements are not envisaged in position 6 of the ring system in WO2011/054844.

The compounds according to the invention differ from the compounds of WO2011/054845 in that the benzene annelated on the diazepine is replaced with thiophene and in that in position 6 of the diazepine an amide residue is provided, which contains at most one ring. Bridged elements or spiro elements are not envisaged in position 6 of the ring system in WO2011/054845.

The compounds according to the invention differ from the nearest prior art 6H-thieno[3,2-f][1,2,4]triazolo[4,3-a][1,4]diazepines that were disclosed as BRD4 inhibitors in WO2009/084693, in that they contain saturated, optionally substituted carbo- or heterocyclic amides with a spiro element and/or a bridged element.

Starting from this prior art, the problem to be solved by the present invention is to provide novel structures for treating human and animal diseases.

In particular, the structures according to the invention should be suitable for the prevention and treatment of tumour diseases and should have advantages over the structures known in the prior art.

In particular, the structures according to the invention should have suitable pharmacokinetics for the prevention and treatment of tumour diseases and should have pharmacokinetic advantages over the structures known in the prior art. Surprisingly, it was found that the compounds according to the invention of formula (I) can possess advantageous pharmacokinetic properties.

Preferably, structures should be made available for treating diseases that in addition also possess one, preferably several or most preferably even all of the following properties:
they are more tolerable in vivo than the structures of the prior art, in particular in the mouse model that is described
they inhibit one or more cancer cell lines more effectively than the structures of the prior art
they have a higher dose-normalized unbound exposure than the structures of the prior art, in particular in the mouse model that is described.

Now, it was found, surprisingly, that compounds of formula (I)

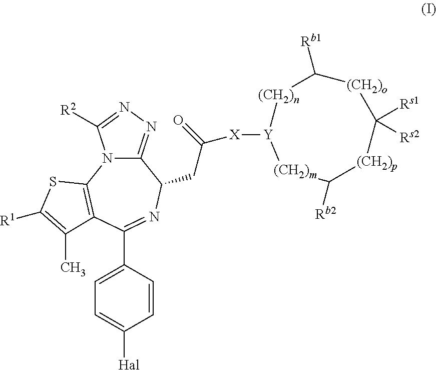

in which
either
X stands for a bond and Y for a nitrogen atom or
X stands for the —NH— group and Y stands for the —CH— group, and
R¹ and R², independently of one another, stand for hydrogen or a C₁-C₆ alkyl group, and
m is 0 or 1, and
n is 0 or 1, and
o is 0 or 1, and
p is 0 or 1,
wherein
the sum of m, n, o and p is at least 2, if R^{b1} and R^{b2} form a bridge, as is defined for compounds of formula (I), and
R^{S1} and R^{S1}, independently of one another, stand for hydrogen or a C₁-C₆ alkyl group, or
R^{S2} together with R^{S1} forms a keto group —C(O)—,
or
R^{S2} together with R^{S1} and the carbon atom to which R^{S1} and R^{S2} are bound, forms a saturated 3- to 8-membered carbocycle or heterocycle, which optionally
   (i) can be substituted one or more times, identically or differently, with halogen, hydroxy, cyano, nitro and/or with a C₁-C₃ alkyl, halo-C₁-C₆ alkyl, C₁-C₆ alkoxy, halo-C₁-C₆ alkoxy, C₁-C₆-alkoxy-C₁-C₆ alkyl and/or C₁-C₆ alkylcarbonyl residue, and/or
   (ii) can contain a keto group —C(O)—, and
R^{b1} and R^{b2} stand for hydrogen, or
R^{b1} and R^{b2} form a bridge consisting of one of the groups
   —O—, —C(O)—, —NR³—, —NR⁴—CHR⁵— or —CHR⁶—CHR⁷—
   wherein R³, R⁴, R⁵, R⁶ and/or R⁷, independently of one another, stand for hydrogen, a C₁-C₆ alkyl or C₁-C₆ alkoxy group or the group —C(O)—R⁸ with R⁸ standing for a C₁-C₆ alkyl or C₁-C₆ alkoxy group
with the proviso,
that either
   R^{b1} and R^{b2} form a bridge, as is defined for compounds of formula (I),
   or
   R^{S2} together with R^{S1} and the carbon atom to which R^{S1} and R^{S2} are bound,
   forms a saturated 3- to 8-membered carbocycle or heterocycle,
or that
   R^{b1} and R^{b2} form a bridge, as is defined for compounds of formula (I), and
   R^{S2} together with R^{S1} and the carbon atom to which R^{S1} and R^{S2} are bound,
   forms a saturated 3- to 8-membered carbocycle or heterocycle,
and the diastereomers, racemates and physiologically compatible salts thereof, are particularly suitable for treating diseases, and solve the problem according to the invention.

As far as the applicant is aware, the structures of the prior art do not have bridged elements or spiro elements in the side chain of position 6 of the ring system.

Starting from the prior art described above, there was no reason to modify the structures of the prior art, since bridged elements and spiro elements in the side chain of position 6 of the ring system are not disclosed for this class of structure, let alone for structures with inhibitory effects on proteins of the BRD family.

Surprisingly, the compounds according to the invention prevent the interaction between BRD4 and an acetylated histone 4 peptide and inhibit the growth of cancer cells. They therefore represent novel structures for treating human and animal diseases, in particular cancers.

The invention is based on the following definitions:

Alkyl:

Alkyl stands for a linear or branched, saturated, monovalent hydrocarbon residue with as a rule 1 to 6 (C₁-C₆ alkyl), preferably 1 to 4 (C₁-C₄ alkyl), and especially preferably 1 to 3 carbon atoms (C₁-C₃ alkyl).

For example and preferably, we may mention:

methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl 1,2-dimethylbutyl.

A methyl, ethyl, propyl or isopropyl residue is especially preferred.

Alkoxy:

Alkoxy stands for a linear or branched, saturated alkylether residue of formula —O-alkyl with as a rule 1 to 6 (C₁-C₆ alkoxy), preferably 1 to 4 (C₁-C₄ alkoxy), and especially preferably 1 to 3 (C₁-C₃ alkoxy) carbon atoms.

For example and preferably, we may mention:

methoxy, ethoxy, n-propoxy, isopropoxy, tert.-butoxy, n-pentoxy and n-hexoxy.

Alkoxyalkyl

Alkoxyalkyl stands for an alkyl residue substituted with alkoxy.

Cₙ-alkoxy-Cₘ-alkyl means that the alkoxy moiety has n carbon atoms and the alkyl moiety, via which the residue is bound, has m carbon atoms.

For example and preferably, we may mention:

methoxymethyl, methoxyethyl, ethoxymethyl and ethoxyethyl.

Alkylcarbonyl

Alkylcarbonyl stands for the —C(O)-alkyl group with as a rule 1 to 6, preferably 1 to 4, and especially preferably 1 to 3 carbon atoms in the alkyl moiety.

We may mention as an example:

Acetyl and propanoyl.

Heteroatoms

Heteroatoms are to be understood as oxygen, nitrogen or sulphur atoms.

Carbocycle:

Carbocycle stands for a monocyclic, saturated, hydrocarbon ring with as a rule 3 to 8 carbon atoms, preferably 4 to 6 carbon atoms.

Heterocycle

Heterocycle stands for a non-aromatic monocyclic ring with 3 to 8 ring atoms, wherein at least one ring atom is a heteroatom or a heterogroup. Nitrogen atoms, oxygen atoms and/or sulphur atoms can be present as heteroatoms. —S(O)—, —S(O)₂— or —N⁺(O⁻)— can be present as heterogroups.

4 to 6 ring atoms are preferred.

Halogen

The designation halogen comprises fluorine, chlorine, bromine and iodine.

Fluorine and chlorine are preferred.

Haloalkyl:

Haloalkyl stands for an alkyl residue with at least one halogen substituent.

For example and preferably, we may mention:

difluoromethyl, trifluoromethyl, 2,2,2-trifluorethyl, pentafluorethyl, 5,5,5,4,4-pentafluoropentyl or 5,5,5,4,4,3,3-heptafluoropentyl.

Perfluorinated alkyl residues such as trifluoromethyl or pentafluorethyl are preferred.

Haloalkoxy

Haloalkoxy stands for an alkoxy residue with at least one halogen substituent.

Fluoroalkoxy residues are preferred.

For example and preferably, we may mention:

difluoroethoxy, trifluomethoxy or 2,2,2-trifluoroethoxy residue.

Cycle

Cycle comprises carbocyclic and heterocyclic rings.

A preferred subgroup is formed by compounds according to formula (I),

in which

either

X stands for a bond and Y for a nitrogen atom or

X stands for the —NH— group and Y stands for the —CH— group, and

R¹ and R², independently of one another, stand for hydrogen or a C₁-C₃ alkyl group, and

m is 0 or 1, and

n is 0 or 1, and

o is 0 or 1, and

p is 0 or 1,

wherein

the sum of m, n, o and p is at least 2, if R^{b1} and R^{b2} form a bridge, as is defined for the preferred subgroup of compounds of formula (I), and

R^{S1} and R^{S1}, independently of one another, stand for hydrogen or a C₁-C₃ alkyl group, or

R^{S2} together with R^{S1} forms a keto group —C(O)—, or

R^{S2} together with R^{S1} and the carbon atom to which R^{S1} and R^{S2} are bound, forms a saturated 4- to 6-membered carbo- or heterocycle, which optionally
(i) can be substituted one or more times, identically or differently, with halogen, hydroxy and/or with a C₁-C₃ alkyl, halo-C₁-C₃ alkyl, C₁-C₃ alkoxy, halo-C₁-C₃ alkoxy, C₁-C₃-alkoxy-C₁-C₃ alkyl and/or C₁-C₃ alkylcarbonyl residue, and/or
(ii) can contain a keto group —C(O)—, and

R^{b1} and R^{b2} stand for hydrogen, or

R^{b1} and R^{b2} form a bridge consisting of one of the groups
—O—, —C(O)—, —NR³—, —NR⁴—CHR⁵— or —CHR⁶—CHR⁷—
wherein R³, R⁴, R⁵, R⁶ and/or R⁷, independently of one another, stand for hydrogen, a C₁-C₃ alkyl or C₁-C₃ alkoxy group or the group —C(O)—R⁸ with R⁸ standing for a C₁-C₄ alkyl or C₁-C₄ alkoxy group

with the proviso,

that either
R^{b1} and R^{b2} form a bridge, as is defined for the preferred subgroup of compounds of formula (I),
or
R^{S2} together with R^{S1} and the carbon atom to which R^{S1} and R^{S2} are bound, forms a saturated 4- to 6-membered carbo- or heterocycle,

or that
R^{b1} and R^{b2} form a bridge, as is defined for the preferred subgroup of compounds of formula (I)
and
R^{S2} together with R^{S1} and the carbon atom to which R^{S1} and R^{S2} are bound, forms a saturated 4- to 6-membered carbo- or heterocycle,

and the diastereomers, racemates and physiologically compatible salts thereof.

A more preferred subgroup is formed by compounds according to formula (I),

in which

either

X stands for a bond and Y for a nitrogen atom or

X stands for the —NH— group and Y stands for the —CH— group, and

R¹ and R² stand for a C₁-C₃ alkyl group, and

m is 0 or 1, and

n is 0 or 1, and

o is 0 or 1, and

p is 0 or 1,

wherein

the sum of m, n, o and p is at least 2, if R^{b1} and R^{b2} form a bridge, as is defined for the more preferred subgroup of compounds of formula (I), and

R^{S1} and R^{S1} stand for hydrogen, or

R^{S2} together with R^{S1} forms a keto group —C(O)—, or

R^{S2} together with R^{S1} and the carbon atom to which R^{S1} and R^{S2} are bound, forms a saturated 4- to 6-membered carbo- or heterocycle with an oxygen atom as heteroatom, which optionally can be substituted one or more times, identically or differently, with halogen, hydroxy and/or with a C₁-C₃ alkyl and/or C₁-C₃-alkoxy residue, and

R^{b1} and R^{b2} stand for hydrogen, or

R^{b1} and R^{b2} form a bridge consisting of one of the groups
—O—, —NR³— or —CHR⁶—CHR⁷—,
wherein R³, R⁶ and/or R⁷ stand for hydrogen or a C₁-C₃ alkyl or C₁-C₃ alkoxy group or the group —C(O)—R⁸ with R⁸ standing for a C₁-C₄ alkyl or C₁-C₄ alkoxy group

with the proviso,

that either
R^{b1} and R^{b2} form a bridge, as is defined for the more preferred subgroup of compounds of formula (I)
or
R^{S2} together with R^{S1} and the carbon atom to which R^{S1} and R^{S2} are bound, forms a saturated 4- to 6-membered carbo- or heterocycle with an oxygen atom as heteroatom

or that
R^{b1} and R^{b2} form a bridge, as is defined for the more preferred subgroup of compounds of formula (I)
and
R^{S2} together with R^{S1} and the carbon atom to which R^{S1} and R^{S2} are bound, forms a
saturated 4- to 6-membered carbo- or heterocycle with an oxygen atom as heteroatom and the diastereomers, racemates and physiologically compatible salts thereof.

A much preferred subgroup is formed by compounds according to formula (I),

in which

X stands for a bond and Y for a nitrogen atom, and

R¹ and R² stand for a methyl group, and

m is 0 or 1, and

n is 0 or 1, and

o is 0 or 1, and

p is 0 or 1,

wherein

the sum of m, n, o and p is at least 2, if R^{b1} and R^{b2} form a bridge, as is defined for the much preferred subgroup of compounds of formula (I), and

R^{S2} together with R^{S1} forms a keto group —C(O)—, or

R^{S2} together with R^{S1} and the carbon atom to which R^{S1} and R^{S2} are bound, forms a saturated 4- to 6-membered heterocycle with an oxygen atom as heteroatom, which optionally can be substituted one or more times, identically or differently, with halogen, hydroxy and/or with a C₁-C₃ alkyl and/or C₁-C₃-alkoxy residue, and

R^{b1} and R^{b2} stand for hydrogen, or

R^{b1} and R^{b2} form a bridge —CHR⁶—CHR⁷—,
wherein R⁶ and/or R⁷ stand for hydrogen or a C₁-C₃ alkyl or C₁-C₃ alkoxy group,

with the proviso,

that either
R^{b1} and R^{b2} form a bridge, as is defined for the much preferred subgroup of compounds of formula (I),
or
R^{S2} together with R^{S1} and the carbon atom to which R^{S1} and R^{S2} are bound, forms a saturated, 4- to 6-membered heterocycle with an oxygen atom as heteroatom, or that
R^{b1} and R^{b2} form a bridge, as is defined for the much preferred subgroup of compounds of formula (I),
and
R^{S2} together with R^{S1} and the carbon atom to which R^{S1} and R^{S2} are bound, forms a saturated, 4- to 6-membered heterocycle with an oxygen atom as heteroatom, and the diastereomers, racemates and physiologically compatible salts thereof.

The following compounds are quite especially preferred:
8-{2-[(S)-4-(4-chlorophenyl)-2,3,9-trimethyl-6H-thieno[3,2-f][1,2,4]triazolo[4,3-a][1,4]diazepin-6-yl]acetyl}-8-azabicyclo[3.2.1]octan-3-one,
2-[(S)-4-(4-chlorophenyl)-2,3,9-trimethyl-6H-thieno[3,2-f][1,2,4]triazolo[4,3-a][1,4]diazepin-6-yl]-1-(2-oxa-6-azaspiro[3.3]hept-6-yl)ethan-1-one,
(1R,5S)-tert-butyl-3-({2-[(S)-4-(4-chlorophenyl)-2,3,9-trimethyl-6H-thieno[3,2-f][1,2,4]triazolo[4,3-a][1,4]diazepin-6-yl]acetyl}amino)-9-azabicyclo[3.3.1]nonane-9-carboxylate,
N-[(1R,5S)-9-azabicyclo[3.3.1]non-3-yl]-2-[(S)-4-(4-chlorophenyl)-2,3,9-trimethyl-6H-thieno[3,2-f][1,2,4]triazolo[4,3-a][1,4]diazepin-6-yl]acetamide,
2-[(S)-4-(4-chlorophenyl)-2,3,9-trimethyl-6H-thieno[3,2-f][1,2,4]triazolo[4,3-a][1,4]diazepin-6-yl]-1-(8-oxa-3-azabicyclo[3.2.1]oct-3-yl)ethan-1-one,
2-[(S)-4-(4-chlorophenyl)-2,3,9-trimethyl-6H-thieno[3,2-f][1,2,4]triazolo[4,3-a][1,4]diazepin-6-yl]-1-(2-oxa-6-azaspiro[3.4]oct-6-yl)ethan-1-one,
2-[(S)-4-(4-chlorophenyl)-2,3,9-trimethyl-6H-thieno[3,2-f][1,2,4]triazolo[4,3-a][1,4]diazepin-6-yl]-1-(2-oxa-7-azaspiro[3.5]non-6-yl)ethan-1-one.

In general formula (I)

either X can stand for a bond and Y for a nitrogen atom or

X for the —NH— group and Y for the —CH— group.

In general formula (I)

X preferably stands for a bond and Y for a nitrogen atom.

In general formula (I)

R¹ and R², independently of one another, can stand for hydrogen or a C₁-C₆ alkyl group.

In general formula (I)

R¹ and R², independently of one another, preferably stand for hydrogen or a C₁-C₃ alkyl group.

In general formula (I)

R¹ and R² more preferably stand for a C₁-C₃ alkyl group.

In general formula (I)

R¹ and R² very preferably stand for a methyl group.

In general formula (I)

m, n, o and p can be 0 or 1,

wherein the sum of m, n, o and p is at least 2, if R^{b1} and R^{b2} form a bridge, as is defined for compounds of formula (I).

In general formula (I)

R^{S1} and R^{S1}, independently of one another, can stand for hydrogen or a C₁-C₆ alkyl group, or

R^{S2} forms, together with R^{S1}, a keto group —C(O)—,

or

R^{S2} forms, together with R^{S1} and the carbon atom to which R^{S1} and R^{S2} are bound, a saturated 3- to 8-membered carbo- or heterocycle, which optionally
(i) can be substituted one or more times, identically or differently, with halogen, hydroxy, cyano, nitro and/or with a C₁-C₃ alkyl, halo-C₁-C₆ alkyl, C₁-C₆ alkoxy, halo-C₁-C₆ alkoxy, C₁-C₆-alkoxy-C₁-C₆ alkyl and/or C₁-C₆ alkylcarbonyl residue and/or
(ii) can contain a keto group —C(O)—.

In general formula (I)

R^{S1} and R^{S1}, independently of one another, preferably stand for hydrogen or a C₁-C₃ alkyl group, or

R^{S2} forms, together with R^{S1}, a keto group —C(O)—, or

R^{S2} forms, together with R^{S1} and the carbon atom to which R^{S1} and R^{S2} are bound, a saturated 4- to 6-membered carbo- or heterocycle, which optionally
(i) can be substituted one or more times, identically or differently, with halogen, hydroxy and/or with a C₁-C₃ alkyl, halo-C₁-C₃ alkyl, C₁-C₃ alkoxy, halo-C₁-C₃ alkoxy, C₁-C₃-alkoxy-C₁-C₃ alkyl and/or C₁-C₃ alkylcarbonyl residue and/or
(ii) can contain a keto group —C(O)—.

In general formula (I)

R^{S1} and R^{S1} more preferably stand for hydrogen, or

R^{S2} forms, together with R^{S1}, a keto group —C(O)—, or

R^{S2} forms, together with R^{S1} and the carbon atom to which R^{S1} and R^{S2} are bound, a saturated 4- to 6-membered carbo- or heterocycle with an oxygen atom as heteroatom, which optionally can be substituted one or more times, identically or differently, with halogen, hydroxy and/or with a C₁-C₃ alkyl and/or C₁-C₃-alkoxy residue.

In general formula (I)

R^{S2} together with R^{S1} very preferably form a keto group —C(O)—, or

R^{S2} together with R^{S1} and the carbon atom to which R^{S1} and R^{S2} are bound, very preferably form a saturated 4- to 6-membered heterocycle with an oxygen atom as heteroatom, which optionally can be substituted one or more times, identically or differently, with halogen, hydroxy and/or with a C₁-C₃ alkyl and/or C₁-C₃-alkoxy residue.

In general formula (I)

R^{b1} and R^{b2} can stand for hydrogen, or

R^{b1} and R^{b2} can form a bridge consisting of one of the groups
—O—, —C(O)—, —NR³—, —NR⁴—CHR⁵— or —CHR⁶—CHR⁷—
wherein R³, R⁴, R⁵, R⁶ and/or R⁷, independently of one another, stand for hydrogen, a C₁-C₆ alkyl or C₁-C₆ alkoxy group or the group —C(O)—R⁸ with R⁸ standing for a C₁-C₆ alkyl or C₁-C₆ alkoxy group.

In general formula (I)

R^{b1} and R^{b2} preferably stand for hydrogen, or

R^{b1} and R^{b2} form a bridge consisting of one of the groups
—O—, —C(O)—, —NR³—, —NR⁴—CHR⁵— or —CHR⁶—CHR⁷—
wherein R³, R⁴, R⁵, R⁶ and/or R⁷, independently of one another, stand for hydrogen, a C₁-C₃ alkyl or C₁-C₃ alkoxy group or the group —C(O)—R⁸ with R⁸ standing for a C₁-C₄ alkyl or C₁-C₄ alkoxy group.

In general formula (I)

R^{b1} and R^{b2} more preferably stand for hydrogen, or

R^{b1} and R^{b2} form a bridge consisting of one of the groups
—O—, —NR³— or —CHR⁶—CHR⁷—,
wherein R³, R⁶ and/or R⁷ stand for hydrogen or a C₁-C₃ alkyl or C₁-C₃ alkoxy group or the group —C(O)—R⁸ with R⁸ standing for a C₁-C₄ alkyl or C₁-C₄ alkoxy group.

In general formula (I)

R^{b1} and R^{b2} very preferably stand for hydrogen, or

R^{b1} and R^{b2} form a bridge —CHR⁶—CHR⁷—,
wherein R⁶ and/or R⁷ stand for hydrogen or a C₁-C₃ alkyl or C₁-C₃ alkoxy group.

The definitions of residues given in detail in the respective combinations or preferred combinations of residues are also replaced arbitrarily with definitions of residues of some other combination independently of the respective combinations of residues stated.

Combinations of two or more of the aforementioned preferred ranges are quite especially preferred.

Compounds according to the invention are the compounds of formula (I) and their salts, solvates and solvates of the salts, the compounds of the formulae stated hereunder covered by formula (I) and their salts, solvates and solvates of the salts and the compounds covered by formula (I) stated hereunder as practical examples and their salts, solvates and solvates of the salts, provided the compounds stated hereunder, covered by formula (I), are not already salts, solvates and solvates of the salts.

The use of the salts of the compounds according to the invention is also to be considered to be covered by the present invention.

Physiologically harmless salts of the compounds according to the invention are preferred as salts in the context of the present invention. However, salts that are not suitable themselves for pharmaceutical uses but can be used for example for isolating or purifying the compounds according to the invention are also included.

Physiologically harmless salts of the compounds according to the invention comprise salts of acid addition of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalene-disulphonic acid, acetic acid, trifluoroacetic acid, propionic acid, lactic acid, tartaric acid, malic acid, citric acid, fumaric acid, maleic acid and benzoic acid.

The present invention further relates to medicinal product containing the compounds according to the invention and at least one or more further active substances, in particular for the prevention and/or treatment of tumour diseases.

Solvates are defined in the context of the invention as those forms of the compounds according to the invention that form a complex in the solid or liquid state by coordination with solvent molecules. Hydrates are a special form of the solvates, for which the coordination takes place with water. Hydrates are preferred as solvates in the context of the present invention.

Depending on their structure, the compounds according to the invention can exist in various stereoisomeric forms, i.e. in the form of configurational isomers or optionally also as conformational isomers. The compounds according to the invention have, in position 6, a uniformly configured asymmetry centre. They can therefore be in the form of pure diastereomers or mixtures thereof, when one or more of the substituents described in formula (I) contains another asymmetry element, for example a chiral carbon atom. The present invention therefore also comprises diastereomers and the respective mixtures thereof. The pure diastereomers can be isolated stereoisomerically in a known way from such mixtures; chromatographic techniques are preferably used for this, in particular HPLC chromatography in achiral or chiral phase.

If the compounds according to the invention can occur in tautomeric forms, the present invention comprises all tautomeric forms.

The present invention also comprises all suitable isotopic variants of the compounds according to the invention. An isotopic variant of a compound according to the invention means a compound in which at least one atom within the compound according to the invention is exchanged for another atom of the same atomic number, but with an atomic mass different from the atomic mass usually or mainly occurring naturally. Examples of isotopes that can be incorporated in a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Certain isotopic variants of a compound according to the invention, such as in particular those in which one or more radioactive isotopes are incorporated, can be useful for example for investigating the mechanism of action or the distribution of the active substance in the body; owing to the comparative ease with which they can be produced and detected, compounds labelled with ³H- or ¹⁴C-isotopes in particular are suitable for this. Furthermore, the incorporation of isotopes, such as for example deuterium, can lead to certain therapeutic advantages as a result of greater metabolic stability of the compound, for example a longer half-life in the body or reduction of the effective dose required; said modifications of the compounds according to the invention can therefore optionally also represent a preferred embodiment of the present invention. Isotopic variants of the compounds according to the invention can be produced by the methods known by a person skilled in the art, for example by the methods described below and according to the specifications presented in the practical examples, using corresponding isotopic modifications of the respective reagents and/or starting compounds.

Moreover, the present invention also comprises prodrugs of the compounds according to the invention. The term “prodrugs” comprises compounds which may themselves be biologically active or inactive, however, during their residence time in the body they are converted (for example metabolically or by hydrolysis) to compounds according to the invention.

The compounds according to the invention can have systemic and/or local action. For this purpose, they can be applied by a suitable method, e.g. oral, parenteral, pulmonary, nasal, sublingual, lingual, buccal, rectal, dermal, transdermal, conjunctival, otic or as implant or stent.

For these routes of administration, the compounds according to the invention can be administered in suitable dosage forms.

Dosage forms suitable for oral application are those functioning according to the prior art for rapid and/or modified release of the compounds according to the invention, containing the compounds according to the invention in crystalline and/or amorphized and/or dissolved form, e.g. tablets (uncoated or coated tablets, for example with enteric coatings or coatings with delayed dissolution or insoluble coatings, which control the release of the compound according to the invention), tablets or films/wafers that disintegrate rapidly in the oral cavity, films/lyophilizates, capsules (for example hard or soft gelatin capsules), sugar-coated pills, granules, pellets, powders, emulsions, suspensions, aerosols or solutions.

Parenteral application can take place with avoidance of an absorption step (e.g. intravenous, intraarterial, intracardiac, intraspinal or intralumbar application) or with inclusion of absorption (e.g. intramuscular, subcutaneous, intracutaneous, percutaneous or intraperitoneal application). Suitable dosage forms for parenteral application include, among others, injection and infusion preparations in the form of solutions, suspensions, emulsions, lyophilizates or sterile powders.

Dosage forms suitable for other routes of administration are for example pharmaceutical forms for inhalation (including powder inhalers, nebulizers), nasal drops, solutions, sprays; tablets for lingual, sublingual or buccal application, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds according to the invention can be transformed into the aforementioned dosage forms. This can take place in a manner known per se by mixing with inert, non-toxic, pharmaceutically suitable excipients. These excipients include, among others, carriers (for example microcrystalline cellulose, lactose, mannitol), solvents (e.g. liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (e.g. antioxidants, such as ascorbic acid), colorants (e.g. inorganic pigments, such as iron oxides) and taste and/or odour correctants.

The present invention further relates to medicinal products that contain the compounds according to the invention, usually together with one or more inert, non-toxic, pharmaceutically suitable excipients, and use thereof for the purposes stated above.

Formulation of the compounds according to the invention to produce pharmaceutical preparations takes place in a manner known per se, wherein the active substance or substances are converted to the desired dosage form with the excipients that are usually employed in pharmaceutical practice.

The excipients that can be used are for example carriers, fillers, disintegrants, binders, humectants, lubricants, absorbents and adsorbents, diluents, solvents, cosolvents, emulsifiers, solubilizers, flavour correctants, colorants, preservatives, stabilizers, wetting agents, salts for altering the osmotic pressure or buffers.

Reference may be made to Remington's Pharmaceutical Science, 15th ed. Mack Publishing Company, East Pennsylvania (1980).

The pharmaceutical formulations can be

in solid form, for example as tablets, sugar-coated pills, pills, suppositories, capsules, transdermal systems or

in semisolid form, for example as ointments, creams, gels, suppositories, emulsions or

in liquid form, for example as solutions, tinctures, suspensions or emulsions.

Excipients in the sense of the invention can be for example salts, saccharides (mono-, di-, tri-, oligo-, and/or polysaccharides), proteins, amino acids, peptides, fats, waxes, oils, hydrocarbons and derivatives thereof, wherein the excipients can be of natural origin or can be obtained synthetically or partially synthetically.

Tablets, sugar-coated pills, capsules, pills, powders, granules, pastilles, suspensions, emulsions or solutions may come into consideration in particular for oral or peroral application.

Suspensions, emulsions and especially solutions may come into consideration in particular for parenteral application.

The present invention relates to the compounds according to the invention.

They can be used for the prevention and treatment of human diseases, in particular of tumour diseases.

The compounds according to the invention can be used in particular to inhibit or reduce cellular proliferation and/or cell division and/or to induce apoptosis.

The compounds according to the invention are suitable in particular for the prevention and/or treatment of hyper-proliferative diseases, for example
psoriasis,
keloid and other hyperplasias that affect the skin,
benign prostatic hyperplasias (BPH),
solid tumours and
haematological tumours.

Solid tumours, treatable according to the invention, are for example tumours of the breast, of the respiratory tract, of the brain, of the reproductive organs, of the gastrointestinal tract, of the urogenital tract, of the eye, of the liver, of the skin, of the head and of the neck, of the thyroid, of the parathyroid, of the bones and of the connective tissue and metastases of these tumours.

As haematological tumours, for example the following are treatable
multiple myelomas,
lymphomas or
leukaemias.

As breast tumours, for example the following are treatable:
breast cancers with positive hormone receptor status
breast cancers with negative hormone receptor status
Her-2 positive breast cancers
hormone-receptor and Her-2 negative breast cancers
BRCA-associated breast cancers
inflammatory breast cancer.

As tumours of the respiratory tract, for example the following are treatable
non-small-cell bronchial carcinomas and
small-cell bronchial carcinomas.

As brain tumours, for example the following are treatable
gliomas,
glioblastomas,
astrocytomas,
meningiomas and
medulloblastomas.

As tumours of the male reproductive organs, for example the following are treatable:
prostate carcinomas,
malignant epididymal tumours,
malignant testicular tumours and
penis carcinomas.

As tumours of the female reproductive organs, for example the following are treatable:
endometrial carcinomas
cervical carcinomas
ovarian carcinomas
vaginal carcinomas
vulvar carcinomas

As tumours of the gastrointestinal tract, for example the following are treatable:
colorectal carcinomas
anal carcinomas
gastric carcinomas
pancreas carcinomas
oesophageal carcinomas
gallbladder carcinomas
small bowel carcinomas
salivary gland carcinomas
neuroendocrine tumours
gastrointestinal stromal tumours

As tumours of the urogenital tract, for example the following are treatable:
bladder carcinomas
renal cell carcinomas
carcinomas of the renal pelvis and of the lower urinary tract

As tumours of the eye, for example the following are treatable:
retinoblastomas
intraocular melanomas

As tumours of the liver, for example the following are treatable:
hepatocellular carcinomas
cholangiocellular carcinomas

As tumours of the skin, for example the following are treatable:
malignant melanomas
basaliomas
prickle-cell carcinomas
Kaposi sarcomas
Merkel cell carcinomas

As tumours of the head and neck, for example the following are treatable:
laryngeal carcinomas
carcinomas of the pharynx and of the oral cavity

As sarcomas, for example the following are treatable:
soft tissue sarcomas
osteosarcomas

As lymphomas, for example the following are treatable:
non-Hodgkin's lymphomas
Hodgkin's lymphomas
cutaneous lymphomas
lymphomas of the central nervous system
AIDS-associated lymphomas

As leukaemias, for example the following are treatable:
acute myeloid leukaemias
chronic myeloid leukaemias
acute lymphatic leukaemias
chronic lymphatic leukaemias
hairy cell leukaemias

Advantageously, the compounds according to the invention can be used for the prevention and/or treatment of leukaemias, in particular acute myeloid leukaemias, prostate carcinomas, in particular androgen receptor-positive prostate carcinomas, cervical carcinomas, breast cancers, in particular hormone receptor-negative, hormone receptor-positive or BRCA-associated breast cancers, pancreas carcinomas, renal cell carcinomas, hepatocellular carcinomas, melanomas and other skin tumours, non-small-cell bronchial carcinomas, endometrial carcinomas and colorectal carcinomas.

The compounds according to the invention can be used especially advantageously for the prevention and/or treatment of acute myeloid leukaemias, prostate carcinomas, in particular androgen receptor-positive prostate carcinomas, cervical carcinomas, breast cancers, in particular oestrogen-alpha-positive and oestrogen-alpha-negative breast cancers, multiple myelomas or melanomas.

The compounds according to the invention are also suitable for the prevention and/or treatment of benign hyperproliferative diseases, for example endometriosis, leiomyoma and benign prostatic hyperplasia.

The compounds according to the invention are also suitable for the prevention and/or treatment of systemic inflammatory diseases, in particular LPS-induced endotoxic shock and/or bacteria-induced sepsis.

The compounds according to the invention are also suitable for the prevention and/or treatment of inflammatory or autoimmune diseases, for example:
lung diseases that are accompanied by inflammatory, allergic and/or proliferative processes: chronic obstructive lung diseases of any origin, especially bronchial asthma; bronchitis of various origins; all forms of restrictive lung diseases, especially allergic alveolitis; all forms of pulmonary oedema, especially toxic pulmonary oedema; sarcoidoses and granulomatoses, in particular Boeck disease
Rheumatic diseases/autoimmune diseases/joint diseases that are accompanied by inflammatory, allergic and/or proliferative processes: all forms of rheumatic diseases, in particular rheumatoid arthritis, acute rheumatic fever, polymyalgia rheumatica; reactive arthritis; inflammatory soft tissue diseases of other origin; arthritic symptoms in degenerative joint diseases (arthroses); traumatic arthritides; collagenoses of any origin, e.g. systemic lupus erythematosus, scleroderma, polymyositis, dermatomyositis, Sjigren syndrome, Still syndrome, Felty syndrome
Allergies that are accompanied by inflammatory and/or proliferative processes: all forms of allergic reactions, e.g. Quincke oedema, hay fever, insect bites, allergic reactions to medicinal products, blood derivatives, contrast media etc., anaphylactic shock, urticaria, contact dermatitis
Vessel inflammations (vasculitides): panarteritis nodosa, temporal arteritis, erythema nodosum
Dermatological diseases that are accompanied by inflammatory, allergic and/or proliferative processes: atopic dermatitis; psoriasis; pityriasis rubra pilaris; erythematous diseases triggered by various noxa, e.g. radiation, chemicals, burns etc.; bullous dermatoses; diseases of the lichenoid type; pruritus; seborrhoeic eczema; rosacea; pemphigus vulgaris; erythema exudativa multiforme; balanitis; vulvitis; hair loss such as alopecia greata; cutaneous T-cell lymphoma
Kidney diseases that are accompanied by inflammatory, allergic and/or proliferative processes: nephrotic syndrome; all nephritides
Liver diseases that are accompanied by inflammatory, allergic and/or proliferative processes: acute liver cell disintegration; acute hepatitis of various origins, e.g. viral, toxic, drug-induced; chronic aggressive and/or chronic intermittent hepatitis
Gastrointestinal diseases that are accompanied by inflammatory, allergic and/or proliferative processes: regional enteritis (Crohn's disease); ulcerative colitis; gastritis; reflux oesophagitis; gastroenteritides of other origin, e.g. coeliac disease
Proctologic diseases that are accompanied by inflammatory, allergic and/or proliferative processes: anal eczema; fissures; haemorrhoids; idiopathic proctitis
Eye diseases that are accompanied by inflammatory, allergic and/or proliferative processes: allergic keratitis, uveitis, iritis; conjunctivitis; blepharitis; neuritis nervi optici; choroiditis; sympathetic ophthalmia
ENT diseases that are accompanied by inflammatory, allergic and/or proliferative processes: allergic rhinitis, hay fever; otitis externa, e.g. caused by contact eczema, infection etc.; otitis media
Neurological diseases that are accompanied by inflammatory, allergic and/or proliferative processes: cerebral oedema, especially tumour-induced cerebral oedema; multiple sclerosis; acute encephalomyelitis; meningitis; various forms of seizures, e.g. salaam seizures
Blood diseases that are accompanied by inflammatory, allergic and/or proliferative processes: acquired haemolytic anaemia; idiopathic thrombocytopenia
Tumour diseases that are accompanied by inflammatory, allergic and/or proliferative processes: acute lymphatic leukaemia; malignant lymphoma; lymphogranulomatoses; lymphosarcomas; extensive metastases, especially in breast, bronchial and prostate carcinoma
Endocrine diseases that are accompanied by inflammatory, allergic and/or proliferative processes: endocrine orbitopathy; thyrotoxic crisis; de Quervain thyroiditis; Hashimoto thyroiditis; Basedow disease
Organ and tissue transplants, graft-versus-host disease
Severe shock, e.g. anaphylactic shock, systemic inflammatory response syndrome (SIRS)
Replacement therapy in: congenital primary adrenal insufficiency, e.g. congenital adrenogenital syndrome; acquired primary adrenal insufficiency, e.g. Addison's disease, autoimmune adrenalitis, postinfectious, tumours, metastases, etc; congenital secondary adrenal insufficiency, e.g. congenital hypopituitarism; acquired secondary adrenal insufficiency, e.g. postinfectious, tumours, etc
Emesis accompanied by inflammatory, allergic and/or proliferative processes, e.g. in combination with a 5-HT3 antagonist in cytostatic-induced vomiting
Pains of inflammatory origin, e.g. lumbago

The compounds according to the invention are also suitable for the treatment of viral diseases, for example infections that are caused by papillomaviruses, herpesviruses, Epstein-Barr viruses, hepatitis B or C viruses, and human immunodeficiency viruses.

The compounds according to the invention are also suitable for the treatment of atherosclerosis, dyslipidaemia, hypercholesterolaemia, hypertriglyceridaemia, peripheral vascular diseases, cardiovascular diseases, angina pectoris, ischaemia, stroke, myocardial infarction, angioplastic restenosis, high blood pressure, thrombosis, adiposity, endotoxaemia.

The compounds according to the invention are also suitable for the treatment of neurodegenerative diseases, for example multiple sclerosis, Alzheimer's disease and Parkinson's disease.

These diseases are well characterized in humans, but also occur in other mammals.

The present application relates to the compounds according to the invention of formula (I), in particular the compounds:
8-{2-[(S)-4-(4-chlorophenyl)-2,3,9-trimethyl-6H-thieno[3,2-f][1,2,4]triazolo[4,3-a][1,4]diazepin-6-yl]acetyl}-8-azabicyclo[3.2.1]octan-3-one,
2-[(S)-4-(4-chlorophenyl)-2,3,9-trimethyl-6H-thieno[3,2-f][1,2,4]triazolo[4,3-a][1,4]diazepin-6-yl]-1-(2-oxa-6-azaspiro[3.3]hept-6-yl)ethan-1-one,
(1R,5S)-tert-butyl-3-({2-[(S)-4-(4-chlorophenyl)-2,3,9-trimethyl-6H-thieno[3,2-f][1,2,4]triazolo[4,3-a][1,4]diazepin-6-yl]acetyl}amino)-9-azabicyclo[3.3.1]nonane-9-carboxylate,
N-[(1R,5S)-9-azabicyclo[3.3.1]non-3-yl]-2-[(S)-4-(4-chlorophenyl)-2,3,9-trimethyl-6H-thieno[3,2-f][1,2,4]triazolo[4,3-a][1,4]diazepin-6-yl]acetamide,
2-[(S)-4-(4-chlorophenyl)-2,3,9-trimethyl-6H-thieno[3,2-f][1,2,4]triazolo[4,3-a][1,4]diazepin-6-yl]-1-(8-oxa-3-azabicyclo[3.2.1]oct-3-yl)ethan-1-one,
2-[(S)-4-(4-chlorophenyl)-2,3,9-trimethyl-6H-thieno[3,2-f][1,2,4]triazolo[4,3-a][1,4]diazepin-6-yl]-1-(2-oxa-6-azaspiro[3.4]oct-6-yl)ethan-1-one,
2-[(S)-4-(4-chlorophenyl)-2,3,9-trimethyl-6H-thieno[3,2-f][1,2,4]triazolo[4,3-a][1,4]diazepin-6-yl]-1-(2-oxa-7-azaspiro[3.5]non-6-yl)ethan-1-one,
(S)-1-(7-azabicyclo[2.2.1]hept-7-yl)-2-[(6S)-4-(4-chlorophenyl)-2,3,9-trimethyl-6H-thieno[3,2-f][1,2,4]triazolo[4,3-a][1,4]diazepin-6-yl]ethanone, —(S)-1-(2-azabicyclo[2,2,2]oct-2-yl)-2-[(6S)-4-(4-chlorophenyl)-2,3,9-trimethyl-6H-thieno[3,2-f][1,2,4]triazolo[4,3-a][1,4]diazepin-6-yl]ethanone.

The present application further relates to the compounds according to the invention for use as medicinal products, in particular for the prevention and/or treatment of tumour diseases.

The present application further relates to the compounds according to the invention for the prevention and/or treatment of leukaemias, in particular acute myeloid leukaemias, prostate carcinomas, in particular androgen receptor-positive prostate carcinomas, cervical carcinomas, breast cancers, in particular hormone receptor-negative, hormone receptor-positive or BRCA-associated breast cancers, pancreas carcinomas, renal cell carcinomas, hepatocellular carcinomas, melanomas and other skin tumours, non-small-cell bronchial carcinomas, endometrial carcinomas and colorectal carcinomas.

The present application further relates to the compounds according to the invention for the prevention and/or treatment of acute myeloid leukaemias, prostate carcinomas, in particular androgen receptor-positive prostate carcinomas, cervical carcinomas, breast cancers, in particular oestrogen-alpha-positive and oestrogen-alpha-negative breast cancers, multiple myelomas or melanomas.

The invention further relates to the use of the compounds according to the invention for preparing a medicinal product.

The present application further relates to the use of the compounds according to the invention for preparing a medicinal product for the prevention and/or treatment of tumour diseases.

The present application further relates to the use of the compounds according to the invention for preparing a medicinal product for the prevention and/or treatment of leukaemias, in particular acute myeloid leukaemias, prostate carcinomas, in particular androgen receptor-positive prostate carcinomas, cervical carcinomas, breast cancers, in particular hormone receptor-negative, hormone receptor-positive or BRCA-associated breast cancers, pancreas carcinomas, renal cell carcinomas, hepatocellular carcinomas, melanomas and other skin tumours, non-small-cell bronchial carcinomas, endometrial carcinomas and colorectal carcinomas.

The present application further relates to the use of the compounds according to the invention for preparing a medicinal product for the prevention and/or treatment of acute myeloid leukaemias, prostate carcinomas, in particular androgen receptor-positive prostate carcinomas, cervical carcinomas, breast cancers, in particular oestrogen-alpha-positive and oestrogen-alpha-negative breast cancers, multiple myelomas or melanomas.

The present application further relates to the use of the compound for the prevention and/or treatment of tumour diseases.

The present application further relates to the use of the compounds according to the invention for the prevention and/or treatment of leukaemias, in particular acute myeloid leukaemias, prostate carcinomas, in particular androgen receptor-positive prostate carcinomas, cervical carcinomas, breast cancers, in particular hormone receptor-negative, hormone receptor-positive or BRCA-associated breast cancers, pancreas carcinomas, renal cell carcinomas, hepatocellular carcinomas, melanomas and other skin tumours, non-small-cell bronchial carcinomas, endometrial carcinomas and colorectal carcinomas.

The present application further relates to the use of the compounds according to the invention for the prevention and/or treatment of acute myeloid leukaemias, prostate carcinomas, in particular androgen receptor-positive prostate carcinomas, cervical carcinomas, breast cancers, in particular oestrogen-alpha-positive and oestrogen-alpha-negative breast cancers, multiple myelomas or melanomas.

The present application further relates to pharmaceutical formulations in the form of tablets containing one of the compounds according to the invention for the prevention and/or treatment of leukaemias, in particular acute myeloid leukaemias, prostate carcinomas, in particular androgen receptor-positive prostate carcinomas, cervical carcinomas, breast cancers, in particular hormone receptor-negative, hormone receptor-positive or BRCA-associated breast cancers, pancreas carcinomas, renal cell carcinomas, hepatocellular carcinomas, melanomas and other skin tumours, non-small-cell bronchial carcinomas, endometrial carcinomas and colorectal carcinomas.

The present application further relates to pharmaceutical formulations in the form of tablets containing one of the compounds according to the invention for the prevention and/or treatment of acute myeloid leukaemias, prostate carcinomas, in particular androgen receptor-positive prostate carcinomas, cervical carcinomas, breast cancers, in particular oestrogen-alpha-positive and oestrogen-alpha-negative breast cancers, multiple myelomas or melanomas.

The invention further relates to the use of the compounds according to the invention for the treatment of diseases that are accompanied by proliferative processes.

The invention further relates to the use of the compounds according to the invention for treating benign hyperplasias, inflammatory diseases, autoimmune diseases, sepsis, viral infections, vascular diseases and neurodegenerative diseases.

The compounds according to the invention can be used alone or if required in combination with one or more other pharmacologically effective substances, provided this combination does not lead to undesirable and unacceptable side-effects. The present invention therefore further relates to medicinal products containing a compound according to the invention and one or more further active substances, in particular for the prevention and/or treatment of the aforementioned diseases.

For example, the compounds according to the invention can be combined with known anti-hyperproliferative, cytostatic or cytotoxic substances for treating cancers. Combining the compounds according to the invention with other usual substances for cancer therapy or also with radiotherapy is especially indicated.

As suitable combination active substances, we may mention for example:

Afinitor, aldesleukin, alendronic acid, Alfaferon, alitretinoin, allopurinol, Aloprim, Aloxi, altretamine, aminoglutethimide, amifostine, amrubicin, amsacrine, anastrozole, Anzemet, Aranesp, Arglabin, arsenic trioxide, Aromasin, 5-azacytidine, azathioprine, BCG or Tice BCG, bestatin, betamethasone acetate, betamethasone sodium phosphate, bexarotene, bleomycin sulphate, broxuridine, bortezomib, busulphan, calcitonin, Campath, capecitabine, carboplatin, Casodex, Cefeson, celmoleukin, Cerubidine, chlorambucil, cisplatin, cladribine, clodronic acid, cyclophosphamide, cytarabine, dacarbazine, dactinomycin, DaunoXome, Decadron, Decadron Phosphate, Delestrogen, denileukin diftitox, Depo-Medrol, deslorelin, dexrazoxane, diethylstilbestrol, Diflucan, docetaxel, doxifluridine, doxorubicin, dronabinol, DW-166HC, Eligard, Elitek, Ellence, emend, epirubicin, epoetin alfa, Epogen, Eptaplatin, Ergamisol, Estrace, estradiol, estramustine phosphate sodium, ethinylestradiol, Ethyol, etidronic acid, Etopophos, etoposide, fadrozole, Fareston, filgrastim, finasteride, floxuridine, fluconazole, fludarabine, 5-fluordeoxyuridine-monophosphate, 5-fluoruracil (5-FU), fluoxymesterone, flutamide, formestane, Fosteabin, fotemustine, fulvestrant, Gammagard, gemcitabine, gemtuzumab, Gleevec, Gliadel, goserelin, granisetron hydrochloride, histrelin, Hycamtin, Hydrocortone, erythro-hydroxynonyladenine, hydroxyurea, ibritumomab tiuxetan, idarubicin, iphosphamide, interferon alfa, interferon alfa-2, interferon alfa-2α, interferon alfa-2β, interferon alfa-n1, interferon alfa-n3, interferon beta, interferon gamma-1α, interleukin-2, Intron A, Iressa, irinotecan, Kytril, lapatinib, lentinan sulphate, letrozole, Leukorganoin, leuprolide, leuprolide acetate, levamisole, Levofolinic acid-calcium salt, Levothroid, Levoxyl, lomustine, lonidamine, Marinol, mechlorethamine, mecobalamin, medroxyprogesterone acetate, megestrol acetate, melphalan, Menest, 6-mercaptopurine, mesna, methotrexate, Metvix, miltefosine, minocycline, mitomycin C, mitotane, mitoxantrone, Modrenal, Myocet, nedaplatin, Neulasta, Neumega, Neupogen, nilutamide, Nolvadex, NSC-631570, OCT-43, octreotide, ondansetron hydrochloride, Orapred, oxaliplatin, paclitaxel, paediapred, pegaspargase, Pegasys, pentostatin, picibanil, pilocarpine hydrochloride, pirarubicin, plicamycin, porfimer sodium, prednimustine, prednisolone, prednisone, Premarin, procarbazine, Procrit, raltitrexed, RDEA119, Rebif, regorafenib, rhenium-186-etidronat, rituximab, Roferon-A, romurtide, Salagen, Sandostatin, sargramostim, semustine, sizofuran, sobuzoxane, Solu-Medrol, streptozocin, strontium-89-chloride, Synthroid, tamoxifen, tamsulosin, tasonermin, testolactone, Taxotere, teceleukin, temozolomide, teniposide, testosterone propionate, Testred, thioguanine, thiotepa, thyrotropin, tiludronic acid, topotecan, toremifene, tositumomab, trastuzumab, treosulfan, tretinoin, Trexall, trimethyl melamine, trimetrexate, triptorelin acetate, triptorelin pamoate, UFT, uridine, valrubicin, vesnarinone, vinblastine, vincristine, vindesine, vinorelbine, Virulizin, Zinecard, zinostatin stimalamer, Zofran; ABI-007, Acolbifene, Actimmune, Affinitak, aminopterin, arzoxifene, asoprisnil, atamestane, atrasentan, BAY 43-9006 (sorafenib), Avastin, CCI-779, CDC-501, Celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin-hydrogel implant, holmium-166-DOTMP, ibandronic acid, interferon gamma, Intron-PEG, ixabepilone, keyhole limpet haemocyanin, L-651582, lanreotide, lasofoxifene, libra, lonafarnib, miproxifene, minodronate, MS-209, liposomal MTP-PE, MX-6, nafarelin, nemorubicin, Neovastat, nolatrexed, oblimersen, onco-TCS, osidem, paclitaxel polyglutamate, pamidronate disodium, PN-401, QS-21, quazepam, R-1549, raloxifene, ranpirnase, 13-cis-retinoic acid, satraplatin, seocalcitol, T-138067, Tarceva, Taxoprexin, thymosin alpha-1, tiazofurin, tipifamib, tirapazamine, TLK-286, toremifene, TransMID-107R, valspodar, vapreotide, vatalanib, verteporfin, vinflunine, Z-100, zoledronic acid, and combinations thereof.

In a preferred embodiment, the compounds according to the invention can be combined with anti-hyperproliferative agents, which may be for example—without this list being exhaustive:

Aminoglutethimide, L-asparaginase, azathioprine, 5-azacytidine, bleomycin, busulphan, carboplatin, carmustine, chlorambucil, cisplatin, colaspase, cyclophosphamide, cytarabine, dacarbazine, dactinomycin, daunorubicin, diethylstilbestrol, 2′,2′-difluordeoxycytidine, docetaxel, doxorubicin (Adriamycin), epirubicin, epothilone and derivatives thereof, erythro-hydroxynonyladenine, ethinylestradiol, etoposide, fludarabine phosphate, 5-fluordeoxyuridine, 5-fluordeoxyuridine monophosphate, 5-fluoruracil, fluoxymesterone, flutamide, hexamethyl melamine, hydroxyurea, hydroxyprogesterone caproate, idarubicin, iphosphamide, interferon, irinotecan, leukorganoin, lomustine, mechlorethamine, medroxyprogesterone acetate, megestrol acetate, melphalan, 6-mercaptopurine, mesna, methotrexate, mitomycin C, mitotane, mitoxantrone, paclitaxel, pentostatin, N-phosphonoacetyl-L-aspartate (PALA), plicamycin, prednisolone, prednisone, procarbazine, raloxifene, semustine, streptozocin, tamoxifen, teniposide, testosterone propionate, thioguanine, thiotepa, topotecan, trimethyl melamine, uridine, vinblastine, vincristine, vindesine and vinorelbine.

Promisingly, the compounds according to the invention can also be combined with biological therapeutics such as antibodies (e.g. Avastin, Rituxan, Erbitux, Herceptin) and recombinant proteins.

The compounds according to the invention can also achieve positive effects in combination with other therapies, directed against angiogenesis, for example with Avastin, axitinib, regorafenib, Recentin, sorafenib or sunitinib. Combinations with proteasome inhibitors and of mTOR and antihormones and steroidal metabolic enzyme inhibitors are particularly suitable owing to their favourable profile of side-effects.

In general, combining the compounds according to the invention with other agents with cytostatic or cytotoxic action can pursue the following objectives:
improved efficacy in slowing tumour growth, in reducing tumour size or even its complete elimination compared to treatment with a single active substance;
the possibility of using the chemotherapeutic agents in lower dosage than in monotherapy;
the possibility of better-tolerated therapy with less side-effects compared to individual administration;
the possibility of treating a wider range of tumour diseases;
attainment of a higher rate of response to the therapy;
longer survival time of the patients in comparison with the current standard therapy.

Furthermore, the compounds according to the invention can also be used in conjunction with radiotherapy and/or surgery.

### 1. Synthesis Routes for Compounds According to Formula (I)

### Description of the Syntheses

The synthesis of tert-butyl[(S)-4-(4-chlorophenyl)-2,3,9-trimethyl-6H-thieno[3,2-f][1,2,4]triazolo[4,3-a][1,4]diazepin-6-yl]acetate has been described (Nature 2010, Vol. 468, p 1067ff, P. Filippakopoulos et al.). The cleavage of the tert-butyl ester can be carried out using strong acids such as trifluoroacetic acid or hydrochloric acid. The example compounds are then obtained by peptide-coupling methods that are known by a person skilled in the art. In these cases (7-aza-1H-benzotriazol-1-yl)-1,1,3,3-tetramethyluronium hexafluorophosphate (HATU) was used as reagent. This is just one example of the reagents that are known by a person skilled in the art (J. American Chem. Soc. 1993, 115, 4397). The variations in each case with respect to R¹, R² and Hal for preparing the carboxylic acids that were used for preparing the compounds according to the invention were described in WO1998/11111. The esters obtained were at that time synthesized as racemates and were cleaved by suitable methods for separation into the enantiomers. HPLC techniques familiar to a person skilled in the art were employed for this, using a chiral stationary phase. Preferably, the respective tert-butyl esters were prepared and were separated into their enantiomers.

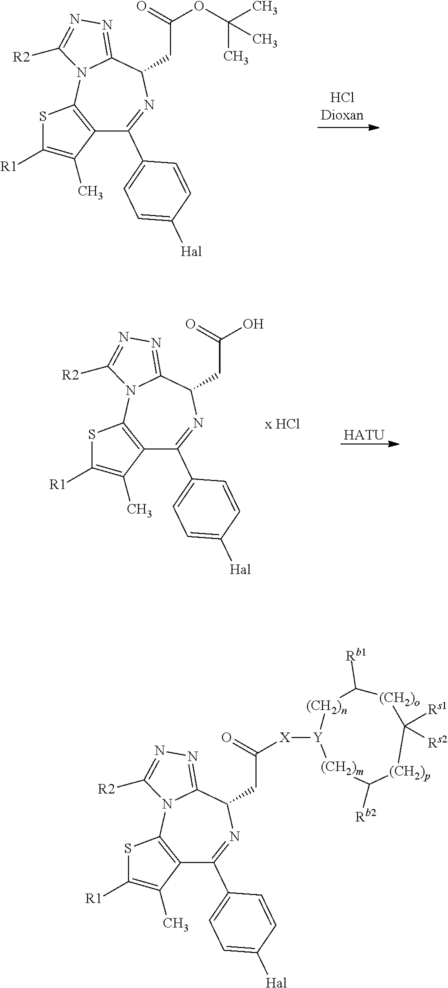

### ABBREVIATIONS AND ACRONYMS

DMF N,N-dimethylformamide
DMSO-d6 deuterated dimethylsulphoxide
DMSO dimethylsulphoxide
HATU (7-aza-1H-benzotriazol-1-yl)-1,1,3,3-tetramethyluronium hexafluorophosphate
RP-HPLC reversed phase high-performance liquid chromatography
RT room temperature
tert tertiary
NMP N-methylpyrrolidone
ACN acetonitrile
HCl hydrochloric acid

### 2. Preparation of the Comparative Examples and Practical Examples

### Starting Compounds and Intermediates

### Precursors

### 6-(Carboxymethyl)-4-(4-chlorophenyl)-2,3,9-trimethyl-6H-thieno[3,2-f][1,2,4]triazolo[4,3-a][1,4]diazepin-8-ium hydrochloride

A solution of 1.6 g (3.5 mmol) of tert-butyl[(S)-4-(4-chlorophenyl)-2,3,9-trimethyl-6H-thieno[3,2-f][1,2,4]triazolo[4,3-a][1,4]diazepin-6-yl]acetate in 25 ml HCl in dioxane (4N) was stirred overnight at RT. The solvent was removed completely under vacuum and the title compound was obtained as a solid. 1.53 g.

¹H-NMR (400 MHz, RT, DMSO-d6): δ=1.6 (s, 3H), 2.39 (s, 3H), 2.61 (s, 3H), 3.31 (dd, 1H), 3.41 (dd, 1H), 4.46 (t, 1H), 4.56 (bs), 7.41 (d, 2H), 7.47 (d, 2H)

### Comparative Example

### tert-Butyl[(S)-4-(4-chlorophenyl)-2,3,9-trimethyl-6H-thieno[3,2-f][1,2,4]triazolo[4,3-a][1,4]diazepin-6-yl]acetate (V1) was used as comparative compound

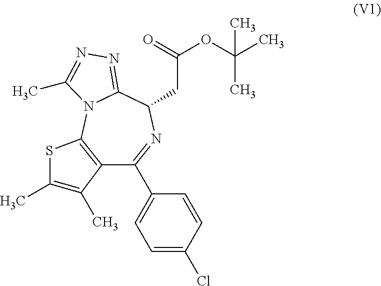 [SMILES: Cc1sc2c(c1C)C(c1ccc(Cl)cc1)=N[C@@H](CC(=O)OC(C)(C)C)c1nnc(C)n1-2]

The preparation of V1 was described in Nature 2010, Vol. 468, p 1067ff (P. Filippakopoulos et al.).

### PRACTICAL EXAMPLES

### Example 1

### 8-{2-[(S)-4-(4-Chlorophenyl)-2,3,9-trimethyl-6H-thieno[3,2-f][1,2,4]triazolo[4,3-a][1,4]diazepin-6-yl]acetyl}-8-azabicyclo[3.2.1]octan-3-one

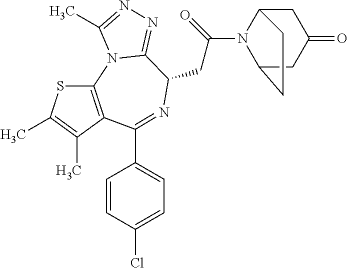 [SMILES: Cc1sc2c(c1C)C(c1ccc(Cl)cc1)=N[C@@H](CC(=O)N1C3CCC1CC(=O)C3)c1nnc(C)n1-2]

0.65 g 2 HATU, 0.4 ml triethylamine and 221.7 mg 3-oxo-8-azoniabicyclo[3.2.1]octane hydrochloride were added to a solution of 0.5 g (1.14 mmol) of (S)-6-(carboxymethyl)-4-(4-chlorophenyl)-2,3,9-trimethyl-6H-thieno[3,2-f][1,2,4]triazolo[4,3-a][1,4]diazepin-8-ium hydrochloride in 10 ml DMF, stirring for 3 hours at RT. Water was added and it was extracted with ethyl acetate. The organic phase was dried over magnesium sulphate and the solvent was removed under vacuum. The title compound was obtained after silica gel chromatography (eluent methylene chloride/methanol gradient) and RP-HPLC (XBridge C18 5 μm 100×30 mm, eluent water/acetonitrile gradient, 0.2% saturated ammonia solution as additive). 0.22 g of the title compound was obtained.

¹H-NMR (300 MHz, RT, CDCl₃): δ=1.67 (d, 3H), 1.70-2.40 (m, 5H), 2.41 (s, 3H), 2.43-2.56 (m, 1H), 2.68 (d, 3H), 2.77 (bdd, 1H), 3.07 (dd, 1H), 3.71 (ddd and d, 1H+1H), 4.77-4.90 (m, 1.5H), 4.90-5.03 (m, 1.5H), 7.28-7.45 (m, 4H)

Opt rotation: [α_{D}]=20.9° (methanol, c=1 g/100 ml)

### Example 2

### 2-[(S)-4-(4-Chlorophenyl)-2,3,9-trimethyl-6H-thieno[3,2-f][1,2,4]triazolo[4,3-a][1,4]diazepin-6-yl]-1-(2-oxa-6-azaspiro[3.3]hept-6-yl)ethan-1-one

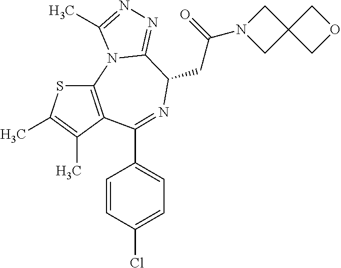 [SMILES: Cc1sc2c(c1C)C(c1ccc(Cl)cc1)=N[C@@H](CC(=O)N1CC3(COC3)C1)c1nnc(C)n1-2]

0.65 g HATU, 0.47 ml triethylamine and 0.2 g di(2-oxa-6-azoniaspiro[3.3]heptane)ethanedioate were added to a solution of 0.5 g (1.14 mmol) of (S)-6-(carboxymethyl)-4-(4-chlorophenyl)-2,3,9-trimethyl-6H-thieno[3,2-f][1,2,4]triazolo[4,3-a][1,4]diazepin-8-ium hydrochloride in 12.5 ml DMF, stirring for 2 hours at RT. Water was added and it was extracted with ethyl acetate. The organic phase was dried over magnesium sulphate and the solvent was removed under vacuum. The title compound was obtained after silica gel chromatography (eluent methylene chloride/methanol gradient) and RP-HPLC (XBridge C18 5 μm 100×30 mm, eluent water/acetonitrile gradient, 0.1% formic acid as additive). 0.13 g of the title compound was obtained.

¹H-NMR (300 MHz, RT, CDCl₃): δ=1.65 (s, 3H), 2.39 (s, 3H), 2.66 (s, 3H), 3.24 (dd, 1H), 3.41 (dd, 1H), 4.2 (s, 2H), 4.52 (d, 1H), 4.68 (t, 1H), 4.73-4.93 (m, 5H), 7.32 (d, 2H), 7.36 (d, 2H)

Opt rotation: [α_{D}]=26.6° (methanol, c=1 g/100 ml)

### Example 3

### (1R,5S)-tert-Butyl-3-({2-[(S)-4-(4-chlorophenyl)-2,3,9-trimethyl-6H-thieno[3,2-f][1,2,4]triazolo[4,3-a][1,4]diazepin-6-yl]acetyl}amino)-9-azabicyclo[3.3.1]nonane-9-carboxylate

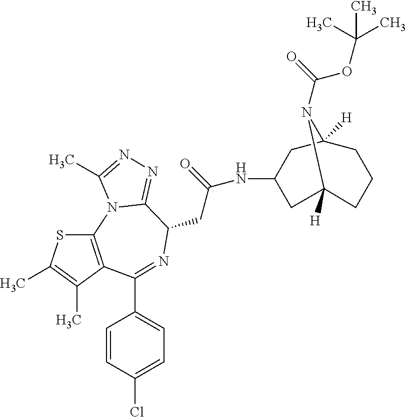 [SMILES: Cc1sc2c(c1C)C(c1ccc(Cl)cc1)=N[C@@H](CC(=O)NC1C[C@H]3CCC[C@@H](C1)N3C(=O)OC(C)(C)C)c1nnc(C)n1-2]

0.39 g HATU, 0.19 ml triethylamine and 0.2 g tert-butyl (1R,5S)-3-amino-9-azabicyclo[3.3.1]nonane-9-carboxylate were added to a solution of 0.3 g (0.69 mmol) of (S)-6-(carboxymethyl)-4-(4-chlorophenyl)-2,3,9-trimethyl-6H-thieno[3,2-f][1,2,4]triazolo[4,3-a][1,4]diazepin-8-ium hydrochloride in 5 ml DMF, stirring for 16 hours at RT. The solution was poured into water, with crystallization of the title compound. After filtration and vacuum drying, 0.35 g of the title compound was obtained.

¹H-NMR (300 MHz, RT, DMSO-d6, selected signals): δ=1.38 (s, 9H), 1.67 (s, 3H), 1.72-1.94 (m, 3H), 2.37 (s, 3H), 2.55 (s, 3H), 3.06-3.23 (m, 2H), 4.14 (bs, 1H), 4.45 (t, 1H), 4.48-4.62 (m, 1H), 7.38 (d, 2H), 7.47 (d, 2H)

### Example 4

### N-[(1R,5S)-9-Azabicyclo[3.3.1]non-3-yl]-2-[(S)-4-(4-chlorophenyl)-2,3,9-trimethyl-6H-thieno[3,2-f][1,2,4]triazolo[4,3-a][1,4]diazepin-6-yl]acetamide

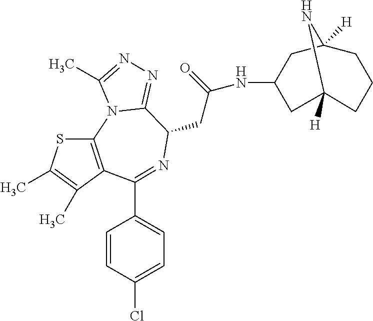 [SMILES: Cc1sc2c(c1C)C(c1ccc(Cl)cc1)=N[C@@H](CC(=O)NC1C[C@H]3CCC[C@@H](C1)N3)c1nnc(C)n1-2]

1 ml of trifluoroacetic acid was added to a solution of 0.35 g (0.56 mmol) of tert-butyl-3-({[2-(S)-4-(4-chlorophenyl)-2,3,9-trimethyl-6H-thieno[3,2-f][1,2,4]triazolo[4,3-a][1,4]diazepin-6-yl]acetyl}amino)-9-azabicyclo[3.3.1]nonane-9-carboxylate in 10 ml dichloromethane, stirring overnight at RT. The reaction mixture was concentrated by vacuum evaporation, water was added and it was made alkaline with saturated sodium carbonate solution. It was extracted with dichloromethane. The solvent was removed under vacuum and the residue was purified by RP-HPLC (XBridge C18 5 μm 100×30 mm, eluent water/acetonitrile gradient, 0.1% formic acid as additive). 12 mg of the title compound was obtained after silica gel chromatography of the raw product (eluent hexane/ethyl acetate gradient).

¹H-NMR (300 MHz, RT, DMSO-d6, selected signals): δ=1.67 (s, 3H), 1.60-1.72 (m, 3H), 1.73-1.98 (m, 3H), 2.38 (s, 3H), 2.56 (s, 3H), 3.06-3.35 (m, 6H), 4.40-4.55 (m, 2H), 7.38 (d, 2H), 7.47 (d, 2H), 7.99 (d, 1/3 H), 8.05 (d, 2/3 H)

### Example 5

### 2-[(S)-4-(4-Chlorophenyl)-2,3,9-trimethyl-6H-thieno[3,2-f][1,2,4]triazolo[4,3-a][1,4]diazepin-6-yl]-1-(8-oxa-3-azabicyclo[3.2.1]oct-3-yl)ethan-1-one

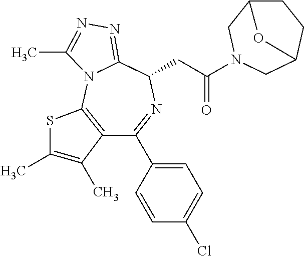 [SMILES: Cc1sc2c(c1C)C(c1ccc(Cl)cc1)=N[C@@H](CC(=O)N1CC3CCC(C1)O3)c1nnc(C)n1-2]

0.199 g HATU, 0.15 ml triethylamine and 0.063 g 8-oxa-3-azabicyclo[3.2.1]octane hydrochloride were added to a solution of 0.14 g (0.35 mmol) of (S)-6-(carboxymethyl)-4-(4-chlorophenyl)-2,3,9-trimethyl-6H-thieno[3,2-f][1,2,4]triazolo[4,3-a][1,4]diazepin-8-ium hydrochloride in 3 ml DMF, stirring overnight at RT. Water was added and it was extracted with ethyl acetate. The organic phase was washed with water and brine. It was dried over sodium sulphate and the solvent was removed under vacuum. The title compound was obtained after silica gel chromatography (eluent methylene chloride/methanol gradient). 0.088 g of the title compound was obtained.

¹H-NMR (300 MHz, RT, CDCl₃): δ=1.67 (s, 3H), 1.7-2.1 (m, 4H), 2.39 (s, 3H), 2.67 (s, 3H), 3.01 (t, 1H), 3.58 (ddd, 1H), 3.52-3.63 (m, 2H), 3.88 (dd, 1H), 4.20 (d, 1H), 4.42 (bs, 2H), 4.82 (t, 1H), 7.32 (d, 2H), 7.40 (dd, 2H)

Opt rotation: [α_{D}]=46.0° (CHCl₃, c=1 g/100 ml)

### Example 6

### 2-[(S)-4-(4-Chlorophenyl)-2,3,9-trimethyl-6H-thieno[3,2-f][1,2,4]triazolo[4,3-a][1,4]diazepin-6-yl]-1-(2-oxa-6-azaspiro[3.4]oct-6-yl)ethan-1-one

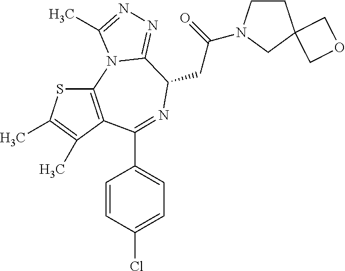 [SMILES: Cc1sc2c(c1C)C(c1ccc(Cl)cc1)=N[C@@H](CC(=O)N1CCC3(COC3)C1)c1nnc(C)n1-2]

0.185 g HATU, 0.14 ml triethylamine and 0.044 g 2-oxa-6-azaspiro[3.4]octane were added to a solution of 0.15 g (0.35 mmol) of (S)-6-(carboxymethyl)-4-(4-chlorophenyl)-2,3,9-trimethyl-6H-thieno[3,2-f][1,2,4]triazolo[4,3-a][1,4]diazepin-8-ium hydrochloride in 5 ml DMF, stirring overnight at RT. Water was added and it was extracted with ethyl acetate. The organic phase was washed with water and brine. It was dried over sodium sulphate and the solvent was removed under vacuum. The title compound was obtained after silica gel chromatography (eluent methylene chloride/methanol gradient). 0.11 g of the title compound was obtained.

¹H-NMR (300 MHz, RT, DMSO-d6): δ=1.67 (s, 3H), 2.09 (t, 1H), 2.22 (t, 1H), 2.37 (s, 3H), 2.55 (d, 3H), 3.20-3.35 (m, 2H), 3.43 (dd, 1H), 3.51 (d, 1H), 3.65 (dt, 1H), 3.90 (dd, 1H), 4.43-4.54 (m, 4H), 4.59 (dd, 1H), 7.38 (dd, 2H), 7.46 (dd, 2H)

Opt rotation: [α_{D}]=30.5° (CHCl₃, c=1 g/100 ml)

### Example 7

### 2-[(S)-4-(4-Chlorophenyl)-2,3,9-trimethyl-6H-thieno[3,2-f][1,2,4]triazolo[4,3-a][1,4]diazepin-6-yl]-1-(2-oxa-7-azaspiro[3.5]non-6-yl)ethan-1-one

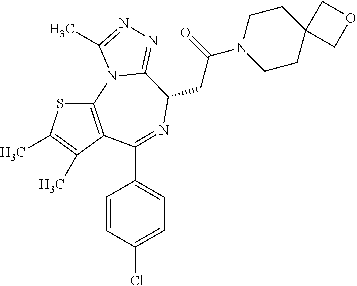 [SMILES: Cc1sc2c(c1C)C(c1ccc(Cl)cc1)=N[C@@H](CC(=O)N1CCC3(CC1)COC3)c1nnc(C)n1-2]

0.185 g HATU, 0.14 ml triethylamine and 0.049 g 2-oxa-7-azaspiro[3.5]nonane were added to a solution of 0.15 g (0.35 mmol) of (S)-6-(carboxymethyl)-4-(4-chlorophenyl)-2,3,9-trimethyl-6H-thieno[3,2-f][1,2,4]triazolo[4,3-a][1,4]diazepin-8-ium hydrochloride in 5 ml DMF, stirring overnight at RT. Water was added and it was extracted with ethyl acetate. The organic phase was washed with water and brine. It was dried over sodium sulphate and the solvent was removed under vacuum. The title compound was obtained after silica gel chromatography (eluent methylene chloride/methanol gradient). 0.115 g of the title compound was obtained.

¹H-NMR (300 MHz, RT, DMSO-d6): δ=1.67 (s, 3H), 1.63-1.71 (m, 2H), 1.81-1.88 (m, 2H), 2.38 (t, 3H), 2.55 (s, 3H), 3.33-3.41 (m, 3H), 3.52 (bt, 2H), 3.58 (dd, 1H), 4.27-4.36 (m, 4H), 4.52 (t, 1H), 7.38 (d, 2H), 7.45 (d, 2H)

Opt rotation: [α_{D}]=37.8° (CHCl₃, c=1 g/100 ml)

### Example 8

### (S)-1-(2-Azabicyclo[2,2,2]oct-2-yl)-2-[(6S)-4-(4-chlorophenyl)-2,3,9-trimethyl-6H-thieno[3,2-j][1,2,4]triazolo[4,3-a][1,4]diazepin-6-yl]ethanone

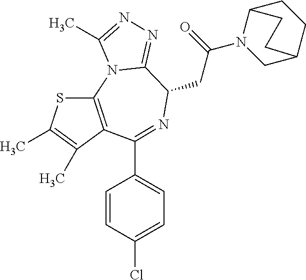 [SMILES: Cc1sc2c(c1C)C(c1ccc(Cl)cc1)=N[C@@H](CC(=O)N1CC3CCC1CC3)c1nnc(C)n1-2]

0.124 g HATU, 0.12 ml triethylamine and 38.5 mg 2-azabicyclo[2,2,2]octane hydrochloride were added to a solution of 100 mg (0.22 mmol) of (S)-6-(carboxymethyl)-4-(4-chlorophenyl)-2,3,9-trimethyl-6H-thieno[3,2-f][1,2,4]triazolo[4,3-a][1,4]diazepin-8-ium hydrochloride in 4.4 ml DMF, stirring overnight at RT. Water was added and it was extracted with ethyl acetate. The organic phase was washed with water and brine. It was dried over sodium sulphate and the solvent was removed under vacuum. The title compound was obtained after silica gel chromatography (eluent methylene chloride/methanol gradient). 46 mg of the title compound was obtained.

¹H-NMR (300 MHz, RT, CDCl₃): δ=1.67 (s, 3H), 1.63-1.78 (m, 6H), 1.80-1.93 (m, 1H); 1.95-2.08 (m, 2H); 2.39 (t, 3H), 2.66 (s, 3H), 3.43-3.63 (m, 3H), 3.81 (dd, 1H); 4.37 (d, 1H); 4.83 (t, 1H), 7.32 (dd, 2H), 7.40 (d, 2H)

### Example 9

### (S)-1-(7-Azabicyclo[2.2.1]hept-7-yl)-2-[(6S)-4-(4-chlorophenyl)-2,3,9-trimethyl-6H-thieno[3,2-f][1,2,4]triazolo[4,3-a][1,4]diazepin-6-yl]ethanone

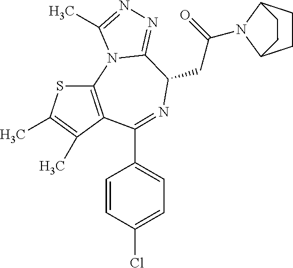 [SMILES: Cc1sc2c(c1C)C(c1ccc(Cl)cc1)=N[C@@H](CC(=O)N1C3CCC1CC3)c1nnc(C)n1-2]

0.124 g HATU, 0.12 ml triethylamine and 34.8 mg 7-azabicyclo[2.2.1]heptane hydrochloride were added to a solution of 100 mg (0.22 mmol) of (S)-6-(carboxymethyl)-4-(4-chlorophenyl)-2,3,9-trimethyl-6H-thieno[3,2-f][1,2,4]triazolo[4,3-a][1,4]diazepin-8-ium hydrochloride in 4.4 ml DMF, stirring overnight at RT. Water was added and it was extracted with ethyl acetate. The organic phase was washed with water and brine. It was dried over sodium sulphate and the solvent was removed under vacuum. The title compound was obtained after silica gel chromatography (eluent methylene chloride/methanol gradient). 50 mg of the title compound was obtained.

¹H-NMR (400 MHz, RT, CDCl₃): δ=1.42-1.63 (s, 4H), 1.67 (s, 3H), 1.74-1.90 (m, 2H), 1.90-2.06 (m, 2H); 2.39 (t, 3H), 2.66 (s, 3H), 3.56 (d, 2H); 4.57 (t, 1H); 4.68 (t, 1H); 4.78 (t, 1H), 7.31 (dd, 2H), 7.39 (d, 2H)

### 3. Assays

### 3.1 Protein-Protein Interaction Assay

### Binding Assay BRD4/Acetylated Peptide H4 (“PRQ”)

To assess the BRD4 binding strength of the substances described in this application, their capacity for dose-dependent inhibition of the interaction between BRD4 and acetylated histone H4 was quantified.

For this purpose, a time-resolved fluorescence resonance energy transfer (TR-FRET) assay was used, which measures the binding between N-terminally His₆-tagged BRD4(1) (amino acids 44-168) and a synthetic acetylated histone H4 (Ac-H4) peptide with the sequence GRGK(Ac)GGK(Ac)GLGK(Ac)GGAK(Ac)RHGSGSK-biotin. The recombinant BRD4 protein (produced in-house) was expressed in E. coli and was purified by (Ni-NTA) affinity chromatography and (Sephadex G-75) size-exclusion chromatography. The Ac-H4 peptide can be purchased for example from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were measured as duplicates on the same microtitre plate. For this, 100-fold concentrated solutions in DMSO were prepared by serial dilutions (1:3.4) of a 2 mM stock solution in a clear, 384-well microtitre plate (Greiner Bio-One, Frickenhausen, Germany). From this, 50 nl was transferred to a black assay plate (Greiner Bio-One, Frickenhausen, Germany). The assay was started by supplying 2 μl of a 2.5-fold concentrated BRD4 solution (usually 10 to 50 nM final concentration in the 5 μl of the reaction volume) in aqueous assay buffer [50 mM HEPES pH 7.5, 50 mM sodium chloride (NaCl), 0.25 mM CHAPS and 0.05% serum albumin (BSA)] to the substances in the assay plate. This was followed by a 10-minute incubation step at 22° C. for the pre-equilibration of putative complexes between BRD4 and the substances. Then 3 μl of a 1.67-fold concentrated solution (in the assay buffer) consisting of Ac-H4 peptide (83.5 nM) and TR-FRET detection reagents [16.7 nM anti-6His-XL665 and 3.34 nM streptavidin cryptate (both from Cisbio Bioassays, Codolet, France), plus 668 mM potassium fluoride (KF)]was added.

The mixture was then incubated in the dark for one hour at 22° C. and then overnight at 4° C. The formation of BRD4/Ac-H4 complexes was determined by measuring the resonance energy transfer from the streptavidin-Eu-cryptate to the anti-6His-XL665 antibody that is present in the reaction. For this, the fluorescence emissions at 620 nm and 665 nm were measured after excitation at 330-350 nm in a TR-FRET measuring instrument, e.g. a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4/Ac-H4 complexes formed.

The data obtained (ratio) were normalized, wherein 0% inhibition corresponded to the mean value from the measured values of a set of controls (usually 32 data points), in which all reagents were contained. 50 nl DMSO (100%) was used instead of test substances. Inhibition of 100% corresponded to the mean value from the measured values of a set of controls (usually 32 data points), in which all reagents except BRD4 were contained. The IC50 value was determined by regression analysis based on a 4-parameter equation (minimum, maximum, IC50, Hill; Y=Max+(Min−Max)/(1+(X/IC50)^{Hill})) using Bayer's own analysis software.

### 3.2 Cell Assays

### Cellular Proliferation Assays

In accordance with the invention, the ability of the substances to inhibit the proliferation of various cell lines was determined. Cell viability was determined by means of the alamarBlue® reagent (Invitrogen). The cells were seeded at different densities (MOLM-13, LAPC-4, MDA-MB-231 and MOLP-8: 4000 cells/well; VCaP: 16000 cells/well; LNCaP: 2000 cells/well; MCF-7 and HeLa-MaTu: 1000 cells/well; B16F10: 400 cells/well) in 100 μl growth medium in 96-well microtitre plates. After incubation overnight at 37° C., the fluorescence values were determined (CI values). Then the plates were treated with various substance dilutions and were incubated at 37° C. for 96 hours (MOLM-13, MCF-7, MDA-MB-231, HeLa-MaTu and B16F10 cells), 120 hours (MOLP-8 cells) or 168 hours (LAPC-4, VCaP and LNCaP cells). Then the fluorescence values were determined (CO values). For data analysis, the CI values were subtracted from the CO values and the results were compared between cells that had been treated with various dilutions of the substance or only with buffer solution. The IC50 values (concentration of substance that is required for 50% inhibition of cellular proliferation) were calculated from these results.

The substances were investigated in the cell lines in Table 1, which for example represent the stated indications:

TABLE 1

| Cell line | Source | Indication |
|---|---|---|
| MOLM-13 | ATCC | acute myeloid leukaemia |
| LAPC-4 | ATCC | prostate carcinoma |
| | | (androgen receptor-positive) |
| VCaP | ATCC | prostate carcinoma |
| | | (androgen receptor-positive) |
| LNCaP | ATCC | prostate carcinoma |
| | | (androgen receptor-positive, T877A mutation) |
| MCF-7 | ATCC | breast cancer |
| | | (oestrogen receptor-alpha positive) |
| MDA-MB-231 | ATCC | breast cancer |
| | | (oestrogen receptor-alpha negative) |
| HeLa-MaTu | ATCC | cervical carcinoma |
| B16F10 | ATCC | melanoma |
| MOLP-8 | ATCC | multiple myeloma |

### 3.3 Determination of Plasma Protein Binding by Equilibrium Dialysis

The binding of test substances to plasma proteins is determined by equilibrium dialysis by means of the Ht-dialysis apparatus (96-well) made of Teflon and a semipermeable membrane (regenerated cellulose, MWCO 12-14K). This separates 150 μl of each of a plasma side and a buffer side (50 mM phosphate buffer). The test substance is added in 2 concentrations (usually 3 and 0.3 μM) to the plasma side and binds to plasma proteins. The unbound fraction of the test substance passes through the membrane and is distributed on either side until equilibrium is established (after approx. 6-8 h at 37° C.). The substance concentration on the buffer side and the plasma side is determined by LC-MS analysis. For this, both sides are brought by dilution with buffer or plasma to the same matrix (10% plasma) and then precipitated with methanol. The free (unbound) fraction (fu) is calculated from the quotient of the buffer and plasma concentration. Stability tests and recovery tests are run concurrently as controls. In addition, the substance in buffer is dialysed against buffer, to check the nonspecific binding to apparatus and membrane and establishment of equilibrium. Because during incubation the osmotic pressure of the plasma proteins leads to dilution of the plasma (volume shift), this possible error is determined by weighing blank plasma samples and is included in the calculation of fu. Establishment of equilibrium and plasma stability should have a value not lower than 80% and the recovery should be at least 30%. A free fraction of <1% is regarded as high, between 1 and 10% as moderate and >10% as low plasma protein binding.

### 3.4 Determination of the Plasma Concentrations from In-Vivo Tests and Calculation of the PK Parameters (via PK Calculation Software, e.g. WinNonLin®)

ACN+internal standard, 1:5 (v/v), was added to mouse plasma, which was obtained at suitable time points after application of the active substance, it was shaken and was frozen out for approx. 12 hours (overnight) at −20° C. After thawing and shaking, the samples are centrifuged for 20 minutes at 4° C. and approx. 2000×g. An aliquot of the supernatant (approx. 25 μL) is measured by LCMS analysis. If high plasma or tissue levels are expected (>ULOQ, as a rule 5 μM) the precipitated samples are additionally diluted 1:100 with ACN/H₂O (80/20, v/v)+internal standard and corresponding aliquots are measured by LCMS. For this, the test substance is added in 5-9 concentrations, corresponding to the determination range of the analytical method, to a control matrix (calibration samples), e.g. 0, 1, 10, 100, 1000, 5000 nM. For this, a portion of solid is weighed and is dissolved in DMSO (as a rule 1 mM stock solution). This stock solution is further diluted 1:10 with DMSO (100 M). Then 1:5 (v/v) ACN+internal standard (soln. A) is added to the calibration samples and processed further as with the plasma samples. The calibration series in solvent takes place similarly to the plasma calibration described. The test substance is in this case prepared in ACN/H₂O (50/50, v/v) and then 1:5 (v/v) ACN+internal standard is added to the samples. This series serves for calibration of the diluted samples. The following PK parameters are calculated from these concentration-time profiles obtained:

AUC₍₀₋ₜₗₐₛₜ₎:

Integrated area under the plasma concentration-time profile from time point zero to the last time point investigated (e.g. 24 h), at which a plasma concentration was measurable.

tlast:

last time point investigated (e.g. 24 h), at which a plasma concentration was measurable.

AUC_{(0-tlast),norm}:

Integrated area under the plasma concentration-time profile from time point zero to the last time point investigated (e.g. 24 h), at which a plasma concentration was measurable, divided by the dose normalized for body weight (in kg*L/h)

AUC_{(0-tlast),norm,u}:

AUC_{(0-tlast),norm} multiplied by the free fraction (fu) of the species investigated.

### 3.5 In-Vivo Tolerability in the Mouse

The substances were formulated in NMP/PEG300 (1/9 V/V). They were administered orally, in an amount of 10 ml/kg, once or twice daily for a period of 5 to 7 days to female NMRI nude mice (6-8 weeks old; 3 animals per group). The dose and the dosage scheme for each substance are shown in the table. Body weight and mortality of the mice were monitored daily up to the end of the study. Toxicity was defined as follows: ≧10% substance-induced deaths or ≧20% weight loss.

### 3.6 In-Vivo Antiproliferative Activity

### 3.6.1 MOLM-13 Acute Monocytic Leukaemia Tumour Model

NMRI nude mice were inoculated subcutaneously in the right flank on day 0 with 2×10⁶ MOLM-13 cells in 0.1 ml Matrigel. Treatment with comparative example V1, and practical examples 1 or 2 was begun on day 3 after tumour inoculation. Comparative example V1 was dissolved in 20% HP beta cyclodextrin in saline (0.2% NaCl in water). Practical example 1 and practical example 2 were dissolved in 40% PEG400, 5% ethanol, 25% Solutol. The substances were administered orally, daily for 11 days (day 3 to day 14). Comparative example V1 was administered at a daily dose of 70 mg/kg (maximum tolerated dose), or 40 mg/kg. Practical examples 1 and 2 were applied at a daily dose of 200 (highest dose used), 120 or 70 mg/kg.

### 3.6.2 B16F10 Melanoma Tumour Model

C57BL/6 mice were inoculated on day 0 with 0.5×10⁶ cells in 0.1 ml medium, subcutaneously, in the right flank. Treatment with comparative example V1 and practical example 2 was begun on day 2 after tumour inoculation. Comparative example V1 was dissolved in 20% HP beta cyclodextrin in saline (0.2% NaCl in water) and practical example 2 in 40% PEG400, 5% ethanol, 25% Solutol. The substances were administered orally for 10 days (day 2 to day 11). Comparative example V1 was applied at a dose of 70 mg/kg (maximum tolerated dose) or 55 mg/kg. Practical example 2 was applied at a dose of 160 or 120 mg/kg.

### 4. Results

### 4.1 Binding Assay

Table 2 shows the results of the binding assay.

TABLE 2

| | HTRF |
|---|---|
| Example | IC50 (nmol/L) |
| 1 | 28 |
| 2 | 27 |
| 3 | 240 |
| 4 | 15 |
| 5 | 78 |
| 6 | 29 |
| 7 | 32 |
| 8 | 84 |
| 9 | 75 |
| V1 (JQ1) | 39 |

### 4.2 Cell Assays

Tables 3a, 3b and 3c show the results of the cellular proliferation assays.

TABLE 3a

| | Leukaemia | Prostate | Prostate | Prostate |
|---|---|---|---|---|
| | MOLM-13 | LAPC-4 | VCaP | LNCaP |
| | IC50 | IC50 | IC50 | IC50 |
| Example | (nmol/L) | (nmol/L) | (nmol/L) | (nmol/L) |
| 1 | 91 | 54 | 39 | 90 |
| 2 | 98 | 43 | 58 | 80 |
| 3 | 83 | 41 | 65 | 17 |
| 4 | 57 | 228 | 71 | 353 |
| 5 | 127 | 65 | 67 | 40 |
| 6 | 131 | 72 | 65 | 36 |
| 7 | 68 | 32 | 35 | 102 |
| 8 | 1060 | 133 | | |
| 9 | 838 | 88 | | |
| V1 (JQ1) | 59 | 39 | 37 | 63 |

TABLE 3b

| | Breast | Breast | Cervix | Melanoma |
|---|---|---|---|---|
| | MCF-7 | MDA-MB-231 | HeLa-MaTu | B16F10 |
| | IC50 | IC50 | IC50 | IC50 |
| Example | (nmol/L) | (nmol/L) | (nmol/L) | (nmol/L) |
| 1 | 146 | 110 | 251 | 97 |
| 2 | 116 | 117 | 202 | 82 |
| 3 | 20 | 129 | 40 | 29 |
| 4 | 300 | 604 | 753 | 1050 |
| 5 | 93 | 138 | 331 | 91 |
| 6 | 98 | 122 | 251 | 87 |
| 7 | 59 | 69 | 167 | 48 |
| 8 | | 363 | | 297 |
| 9 | | 291 | | 414 |
| V1 (JQ1) | 148 | 86 | 107 | 67 |

TABLE 3c

| | Multiple |
|---|---|
| | myeloma |
| | MOLP-8 |
| Example | IC50 (nmol/L) |
| 1 | 70 |
| 2 | 64 |
| 3 | 170 |
| 4 | 240 |
| 5 | 120 |
| 6 | 110 |
| 7 | 51 |
| 8 | |
| 9 | |
| V1 (JQ1) | 63 |

### 4.3 Plasma Protein Binding by Equilibrium Dialysis

Table 4 shows the results from determination of plasma protein binding.

TABLE 4

| Example | Protein binding, given as % fu |
|---|---|
| V1 | 1.5 |
| 1 | 12 |
| 2 | 25 |

### 4.4. Plasma Concentrations from In-Vivo Tests and PK Parameters (Via PK Calculation Software, e.g. WinNonLin®)

Table 5 shows the plasma concentrations determined in the in-vivo test (mouse) and Table 6 shows the pharmacokinetic parameters determined.

TABLE 5

| Example | 1 | | 2 | V1 |
|---|---|---|---|---|
| Applied dose | 100 | | 50 | 60 |
| [mg/kg] (p.o.) | | | | |
| | Concentrations in [ng/mL] | | | |
| l h | 1473 | ng/mL | 1366.5 ng/mL | 2245.8 ng/mL |
| 3 h | 835.7 | ng/mL | 2694.4 ng/mL | 1220.2 ng/mL |
| 5 h | | | | 587.2 ng/mL |
| 6 h | 176.3 | ng/mL | 1111.0 ng/mL | |
| 7 h | | | | 321.5 ng/mL |
| 24 h | 1.56 | ng/mL | | |

TABLE 6

| | | 1 | 2 | V1 |
|---|---|---|---|---|
| Dose | (mg/kg) | 100 | 50 | 60 |
| AUC₍₀₋ₜₗₐₛₜ₎. | (mg*h/kg) | 4.9 | 10 | 7.1 |
| tlast | (h) | 24 | 6.0 | 7.0 |
| AUC_{(0-tlast),norm} | (kg*h/L) | 0.05 | 0.20 | 0.12 |
| fu | (in %) | 12 | 25 | 1.5 |
| AUC_{(0-tlast),norm,u} | (kg*h/L) | 0.006 | 0.050 | 0.002 |

AUC_{(0-tlast),norm,u} indicates that examples 1 and 2 according to the invention in comparison with comparative example V1 in the effective species mouse have a higher unbound exposure after single oral administration. In the mouse, there are therefore higher dose-normalized free plasma concentrations, so that at equal dose, an increased efficacy is to be expected in the mouse.

### 4.5 In-Vivo Tolerability in the Mouse

Table 7 shows the results from the in-vivo tolerability test (mouse).

Comparative substance V1 was tolerated at a daily dose of 100 mg/kg for the 7 days. The weight loss was highest at 10% on the 9th day of treatment. With twice daily administration of 100 mg/kg the substance was not tolerated, as 2 substance-induced deaths were observed on the 6th day of treatment. The maximum tolerated treatment dose (MTD) after 5 days was 50 mg/kg twice daily, with a maximum body weight loss of 7% on day 6.

Practical examples 1 and 2 were well tolerated at all doses tested in treatment once or twice daily. The maximum tolerated treatment dose was ≧200 mg/kg daily or ≧100 mg/kg twice daily after 5 days of treatment. The body weight loss was less than 3% in all groups.

To summarize, practical examples 1 and 2 showed better tolerability in mice than comparative substance V1. The maximum tolerated treatment dose in single daily treatment was ≧200 mg/kg for practical examples 1 and 2, and 100 mg/kg for the comparative substance V1. The maximum tolerated treatment dose in twice daily administration was ≧100 mg/kg for practical examples 1 and 2, and 50 mg/kg for the comparative substance V1.

TABLE 7

| | Oral | Dosage | % maximum body weight | Drug-induced | |
|---|---|---|---|---|---|
| Example | dose (mg/kg) | scheme | change (day) | deaths(day) | Remarks |
| V1 | 100 | QD × 7, | −10 (9) | 0/3 | MTD (10% body- |
| | | once daily | | | weight loss) |
| | 100 | QD × 5, | −19 (5) | 2/3 (6) | Not tolerated |
| | 50 | twice daily | −7 (6) | 0/3 | MTD |
| 1 | 200 | QD × 5, | +5 (6) | 0/3 | HDT tolerated |
| | 100 | once daily | +6 (6) | 0/3 | tolerated |
| | 50 | | +7 (6) | 0/3 | tolerated |
| | 100 | QD × 5, | −1 (6) | 0/3 | HDT tolerated |
| | 50 | twice daily | +5 (4) | 0/3 | tolerated |
| 2 | 200 | QD × 5, | +4 (4) | 0/3 | HDT tolerated |
| | 100 | once daily | −1 (3) | 0/3 | tolerated |
| | 50 | | +5 (6) | 0/3 | tolerated |
| | 100 | QD × 5, | −3 (5) | 0/3 | HDT tolerated |
| | 50 | twice daily | +2 (6) | 0/3 | tolerated |

MTD = maximum tolerated treatment dose, HDT = highest dose tested

### 4.6 In-Vivo Antiproliferative Action

### 4.6.1 MOLM-13 Acute Monocytic Leukaemia Tumour Model

The animals' weight increased during the study. There was one unexplained death in the group that was treated with practical example 2.

The highest dose of comparative example V1 was biologically active, as 20% T/C was measured on day 14. The low dose was inactive and had a T/C value of 54%. The highest dose (200 mg/kg) of practical example 1 inhibited tumour growth (T/C value 39%), the 120 mg/kg dose also showed activity (T/C value 46%) and the lowest dose was inactive (T/C value 58%). The highest dose (200 mg/kg) of practical example 2 was active and had a T/C value of 23%. Lower doses (120 and 70 mg/kg) also showed effects on tumour growth (T/C value 46%), but these were not statistically significant. Statistical significance is defined as P<0.05.

### 4.6.2 B16F10 Melanoma Tumour Model

Treatment with comparative example V1 led to a weight loss of 6 or 2% at the 160 or 120 mg/kg dose, respectively. Practical example 2 led to a weight loss of 5 or 2% at the 160 or 120 mg/kg dose, respectively. One mouse (out of 12) died on day 12 in both groups that were treated with comparative example V1. In the group that was treated with 70 mg/kg of practical example 2, one mouse, which had weight loss above 20%, had to be killed on day 10. For the highest dose of both substances, on some days the treatment had to be stopped, as some mice showed weight loss of more than 10%. The highest tolerated dose (MTD) was 55 mg/kg for comparative example V1 and 120 mg/kg for practical example 2. At these doses, both substances were significantly active. Comparative example V1 showed a T/C value of 33% and practical example 2 showed a T/C value of 27%.

1. Compounds of formula (I) 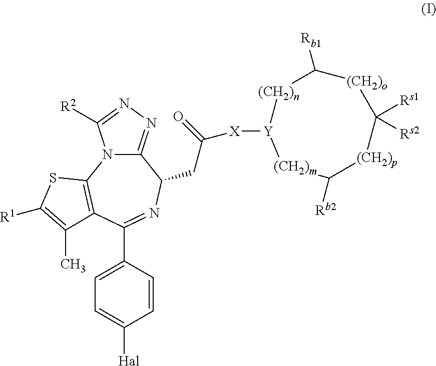
in which
either
X stands for a bond and Y for a nitrogen atom or
X stands for the —NH— group and Y stands for the —CH— group, and
R¹ and R², independently of one another, stand for hydrogen or a C₁-C₆ alkyl group, and
m is 0 or 1, and
n is 0 or 1, and
o is 0 or 1, and
p is 0 or 1,
wherein
the sum of m, n, o and p is at least 2, if R^{b1} and R^{b2} form a bridge, and
R^{S1} and R^{S1}, independently of one another, stand for hydrogen or a C₁-C₆ alkyl group, or
R^{S2} together with R^{S1} forms a keto group —C(O)—,
or
R^{S2} together with R^{S1} and the carbon atom to which R^{S1} and R^{S2} are bound, forms a saturated 3- to 8-membered carbocycle or heterocycle, which optionally
(i) can be substituted one or more times, identically or differently, with halogen, hydroxy, cyano, nitro and/or with a C₁-C₃ alkyl, halo-C₁-C₆ alkyl, C₁-C₆ alkoxy, halo-C₁-C₆ alkoxy, C₁-C₆-alkoxy-C₁-C₆ alkyl and/or C₁-C₆ alkylcarbonyl residue, and/or
(ii) can contain a keto group —C(O)—, and
R^{b1} and R^{b2} stand for hydrogen, or
R^{b1} and R^{b2} form a bridge consisting of one of the groups
—O—, —C(O)—, —NR³—, —NR⁴—CHR⁵— or —CHR⁶—CHR⁷—
wherein R³, R⁴, R⁵, R⁶ and/or R⁷, independently of one another, stand for hydrogen, a C₁-C₆ alkyl or C₁-C₆ alkoxy group or the group —C(O)—R⁸ with R⁸ standing for a C₁-C₆ alkyl or C₁-C₆ alkoxy group
with the proviso,
that either
R^{b1} and R^{b2} form a bridge,
or
R^{S2} together with R^{S1} and the carbon atom to which R^{S1} and R^{S2} are bound, forms a saturated 3- to 8-membered carbocycle or heterocycle,
or that
R^{b1} and R^{b2} form a bridge,
and
R^{S2} together with R^{S1} and the carbon atom to which R^{S1} and R^{S2} are bound, forms a saturated 3- to 8-membered carbocycle or heterocycle,
and the diastereomers, racemates and physiologically compatible salts thereof.

2. Compounds according to claim 1, wherein
X stands for a bond and Y for a nitrogen atom,
and the diastereomers, racemates and physiologically compatible salts thereof.

3. Compounds according to claim 1, wherein
R¹ and R² stand for a methyl group,
and the diastereomers, racemates and physiologically compatible salts thereof.

4. Compounds according to claim 1, wherein
R^{S2} together with R^{S1} forms a keto group —C(O)—, or
R^{S2} together with R^{S1} and the carbon atom to which R^{S1} and R^{S2} are bound, forms a saturated 4- to 6-membered heterocycle with an oxygen atom as heteroatom, which optionally can be substituted one or more times, identically or differently, with halogen, hydroxy and/or with a C₁-C₃ alkyl and/or C₁-C₃-alkoxy residue,
and the diastereomers, racemates and physiologically compatible salts thereof.

5. Compounds according to claim 1, wherein
R^{b1} and R^{b2} stand for hydrogen, or
R^{b1} and R^{b2} form a bridge —CHR⁶—CHR⁷—,
wherein R⁶ and/or R⁷ stand for hydrogen or a C₁-C₃ alkyl or C₁-C₃ alkoxy group, and the diastereomers, racemates and physiologically compatible salts thereof.

6. Compounds according to claim 1,
in which
either
X stands for a bond and Y for a nitrogen atom or
X stands for the —NH— group and Y stands for the —CH— group, and
R¹ and R² stand for a C₁-C₃ alkyl group, and
m is 0 or 1, and
n is 0 or 1, and
o is 0 or 1, and
p is 0 or 1,
wherein
the sum of m, n, o and p is at least 2, if R^{b1} and R^{b2} form a bridge, and
R^{S1} and R^{S1} stand for hydrogen, or
R^{S2} together with R^{S1} forms a keto group —C(O)—, or
R^{S2} together with R^{S1} and the carbon atom to which R^{S1} and R^{S2} are bound, forms a saturated 4- to 6-membered carbo- or heterocycle with an oxygen atom as heteroatom, which optionally can be substituted one or more times, identically or differently, with halogen, hydroxy and/or with a C₁-C₃ alkyl and/or C₁-C₃-alkoxy residue, and
R^{b1} and R^{b2} stand for hydrogen, or
R^{b1} and R^{b2} form a bridge consisting of one of the groups
—O—, —NR³— or —CHR⁶—CHR⁷—,
wherein R³, R⁶ and/or R⁷ stand for hydrogen or a C₁-C₃ alkyl or C₁-C₃ alkoxy group or the group —C(O)—R⁸ with R⁸ standing for a C₁-C₄ alkyl or C₁-C₄ alkoxy group
with the proviso,
that either
R^{b1} and R^{b2} form a bridge,
or
R^{S2} together with R^{S1} and the carbon atom to which R^{S1} and R^{S2} are bound, forms a saturated 4- to 6-membered carbo- or heterocycle with an oxygen atom as heteroatom,
or that
R^{b1} and R^{b2} form a bridge,
and
R^{S2} together with R^{S1} and the carbon atom to which R^{S1} and R^{S2} are bound, forms a saturated 4- to 6-membered carbo- or heterocycle with an oxygen atom as heteroatom,
and the diastereomers, racemates and physiologically compatible salts thereof.

7. Compounds according to claim 1,
in which
X stands for a bond and Y for a nitrogen atom, and
R¹ and R² stand for a methyl group, and
m is 0 or 1, and
n is 0 or 1, and
o is 0 or 1, and
p is 0 or 1,
wherein
the sum of m, n, o and p is at least 2, if R^{b1} and R^{b2} form a bridge, and
R^{S2} together with R^{S1} forms a keto group —C(O)—, or
R^{S2} together with R^{S1} and the carbon atom to which R^{S1} and R^{S2} are bound, forms a saturated 4- to 6-membered heterocycle with an oxygen atom as heteroatom, which optionally can be substituted one or more times, identically or differently, with halogen, hydroxy and/or with a C₁-C₃ alkyl and/or C₁-C₃-alkoxy residue, and
R^{b1} and R^{b2} stand for hydrogen, or
R^{b1} and R^{b2} form a bridge —CHR⁶—CHR⁷—,
wherein R⁶ and/or R⁷ stand for hydrogen or a C₁-C₃ alkyl or C₁-C₃ alkoxy group,
with the proviso,
that either
R^{b1} and R^{b2} form a bridge, as is defined according to this claim for compounds of formula (I),
or
R^{S2} together with R^{S1} and the carbon atom to which R^{S1} and R^{S2} are bound, forms a saturated, 4- to 6-membered heterocycle with an oxygen atom as heteroatom,
or that
R^{b1} and R^{b2} form a bridge, as is defined according to this claim for compounds of formula (I),
and
R^{S2} together with R^{S1} and the carbon atom to which R^{S1} and R^{S2} are bound, forms a saturated, 4- to 6-membered heterocycle with an oxygen atom as heteroatom,
and the diastereomers, racemates and physiologically compatible salts thereof.

8. A compound according to claim 1, which is selected from the group consisting of
8-{2-[(S)-4-(4-chlorophenyl)-2,3,9-trimethyl-6H-thieno[3,2-f][1,2,4]triazolo[4,3-a][1,4]diazepin-6-yl]acetyl}-8-azabicyclo[3.2.1]octan-3-one,
2-[(S)-4-(4-chlorophenyl)-2,3,9-trimethyl-6H-thieno[3,2-f][1,2,4]triazolo[4,3-a][1,4]diazepin-6-yl]-1-(2-oxa-6-azaspiro[3.3]hept-6-yl)ethan-1-one,
(1R,5S)-tert-butyl-3-({2-[(S)-4-(4-chlorophenyl)-2,3,9-trimethyl-6H-thieno[3,2-f][1,2,4]triazolo[4,3-a][1,4]diazepin-6-yl]acetyl}amino)-9-azabicyclo[3.3.1]nonane-9-carboxylate,
N-[(1R,5S)-9-azabicyclo[3.3.1]non-3-yl]-2-[(S)-4-(4-chlorophenyl)-2,3,9-trimethyl-6H-thieno[3,2-f][1,2,4]triazolo[4,3-a][1,4]diazepin-6-yl]acetamide,
2-[(S)-4-(4-chlorophenyl)-2,3,9-trimethyl-6H-thieno[3,2-f][1,2,4]triazolo[4,3-a][1,4]diazepin-6-yl]-1-(8-oxa-3-azabicyclo[3.2.1]oct-3-yl)ethan-1-one,
2-[(S)-4-(4-chlorophenyl)-2,3,9-trimethyl-6H-thieno[3,2-f][1,2,4]triazolo[4,3-a][1,4]diazepin-6-yl]-1-(2-oxa-6-azaspiro[3.4]oct-6-yl)ethan-1-one,
2-[(S)-4-(4-chlorophenyl)-2,3,9-trimethyl-6H-thieno[3,2-f][1,2,4]triazolo[4,3-a][1,4]diazepin-6-yl]-1-(2-oxa-7-azaspiro[3.5]non-6-yl)ethan-1-one,
S)-1-(7-azabicyclo[2.2.1]hept-7-yl)-2-[(6S)-4-(4-chlorophenyl)-2,3,9-trimethyl-6H-thieno[3,2-f][1,2,4]triazolo[4,3-a][1,4]diazepin-6-yl]ethanone, and
(S)-1-(2-azabicyclo[2,2,2]oct-2-yl)-2-[(6S)-4-(4-chlorophenyl)-2,3,9-trimethyl-6H-thieno[3,2-f][1,2,4]triazolo[4,3-a][1,4]diazepin-6-yl]ethanone.

9. (canceled)

10. (canceled)

11. (canceled)

12. (canceled)

13. (canceled)

14. (canceled)

15. (canceled)

16. A method for the prevention and/or treatment of a disease selected from tumour diseases, benign hyperplasias, inflammatory diseases, autoimmune diseases, sepsis, viral infections, vascular diseases and neurodegenerative diseases comprising administering to a patient in need thereof a therapeutically effective amount of a compound according to claim 1 or a diastereomer, racemate or physiologically compatible salt thereof.

17. The method according to claim 16 wherein the disease is selected from acute myeloid leukaemias, prostate carcinomas, cervical carcinomas, breast cancers, multiple myelomas and melanomas.

18. A pharmaceutical composition comprising a compound according to claim 1, or a diastereomer, racemate or physiologically compatible salt thereof, in combination with another active substance.

19. A pharmaceutical composition comprising a compound according to claim 1, or a diastereomer, racemate or physiologically compatible salt thereof, in combination with a pharmaceutically acceptable carrier.
